SEQUENCE LISTING (34 sequences)

SEQ ID NO: 1 - RNA, 23 nt, synthetic construct
  source 1..23: mol_type = other RNA
caaagtgcttacagtgcaggtag

SEQ ID NO: 2 - RNA, 23 nt, synthetic construct
  source 1..23: mol_type = other RNA
ctacctgcactgtaagcactttg

SEQ ID NO: 3 - DNA, 15 bp, synthetic construct
  source 1..15: mol_type = other DNA
  modified_base 1..15: mod_base = OTHER; note = phosphorothioate internucleoside linkage
  modified_base 2: mod_base = OTHER; note = locked nucleic acid base is Adenosine
  modified_base 4: mod_base = OTHER; note = locked nucleic acid base is Thymine
  modified_base 6: mod_base = OTHER; note = locked nucleic acid base is Thymine
  modified_base 9: mod_base = OTHER; note = locked nucleic acid base is Guanine
  modified_base 10: mod_base = OTHER; note = locked nucleic acid base is Cytosine
  modified_base 13: mod_base = OTHER; note = locked nucleic acid base is Thymine
  modified_base 15: mod_base = OTHER; note = locked nucleic acid base is Thymine
  modified_base 11: mod_base = OTHER; note = deoxyadenosine
cactgtaagcacttt

SEQ ID NO: 4 - DNA, 15 bp, synthetic construct
  source 1..15: mol_type = other DNA
  modified_base 1..12: mod_base = OTHER; note = phosphorothioate internucleoside linkage
  modified_base 1: mod_base = OTHER; note = locked nucleic acid base is Cytosine
  modified_base 2: mod_base = OTHER; note = locked nucleic acid base is Thymine
  modified_base 4: mod_base = OTHER; note = locked nucleic acid base is Adenosine
  modified_base 6: mod_base = OTHER; note = locked nucleic acid base is Thymine
  modified_base 9: mod_base = OTHER; note = locked nucleic acid base is Adenosine
  modified_base 10: mod_base = OTHER; note = locked nucleic acid base is Guanine
  modified_base 13: mod_base = OTHER; note = locked nucleic acid base is Adenosine
  modified_base 15: mod_base = OTHER; note = locked nucleic acid base is Thymine
  modified_base 13..15: mod_base = OTHER; note = phosphorothioate internucleoside linkage
ctcagtaaagatatt

SEQ ID NO: 5 - DNA, 15 bp, synthetic construct
  source 1..15: mol_type = other DNA
cactgtaagcacttt

SEQ ID NO: 6 - DNA, 15 bp, synthetic construct
  source 1..15: mol_type = other DNA
ctcagtaaagatatt

SEQ ID NO: 7 - DNA, 23 bp, synthetic construct
  source 1..23: mol_type = other DNA
ctacctgcactgtaagcactttg

SEQ ID NO: 8 - DNA, 23 bp, synthetic construct
  source 1..23: mol_type = other DNA
ctacctccagtgaaagctctatg

SEQ ID NO: 9 - RNA, 23 nt, synthetic construct
  source 1..23: mol_type = other RNA
taaggtgcatctagtgcagatag

SEQ ID NO: 10 - RNA, 23 nt, synthetic construct
  source 1..23: mol_type = other RNA
ctatctgcactagatgcacctta

SEQ ID NO: 11 - DNA, 23 bp, synthetic construct
  source 1..23: mol_type = other DNA
ctatctgcactagatgcacctta

SEQ ID NO: 12 - DNA, 23 bp, synthetic construct
  source 1..23: mol_type = other DNA
ctatctccagtacatggacgtta

SEQ ID NO: 13 - RNA, 22 nt, synthetic construct
  source 1..22: mol_type = other RNA
agttttgcatagttgcactaca

SEQ ID NO: 14 - RNA, 22 nt, synthetic construct
  source 1..22: mol_type = other RNA
tcaaaacgtatcaacgtgatgt

SEQ ID NO: 15 - DNA, 22 bp, synthetic construct
  source 1..22: mol_type = other DNA
tcaaaacgtatcaacgtgatgt

SEQ ID NO: 16 - DNA, 22 bp, synthetic construct
  source 1..22: mol_type = other DNA
tctaatcgaatgatcgagttgt

SEQ ID NO: 17 - RNA, 23 nt, synthetic construct
  source 1..23: mol_type = other RNA
taaagtgcttatagtgcaggtag

SEQ ID NO: 18 - RNA, 23 nt, synthetic construct
  source 1..23: mol_type = other RNA
ctacctgcactataagcacttta

SEQ ID NO: 19 - DNA, 23 bp, synthetic construct
  source 1..23: mol_type = other DNA
ctacctgcactataagcacttta

SEQ ID NO: 20 - DNA, 23 bp, synthetic construct
  source 1..23: mol_type = other DNA
cttccagcagtaaaaccagtata

SEQ ID NO: 21 - RNA, 23 nt, synthetic construct
  source 1..23: mol_type = other RNA
agttttgcaggtttgcatccagc

SEQ ID NO: 22 - RNA, 23 nt, synthetic construct
  source 1..23: mol_type = other RNA
gctggatggaaacctgcaaaact

SEQ ID NO: 23 - DNA, 23 bp, synthetic construct
  source 1..23: mol_type = other DNA
gctggatggaaacctgcaaaact

SEQ ID NO: 24 - DNA, 23 bp, synthetic construct
  source 1..23: mol_type = other DNA
gcaggaagcaaagctcctaaact

SEQ ID NO: 25 - RNA, 23 nt, synthetic construct
  source 1..23: mol_type = other RNA
aggttgggatcggttgcaatgct

SEQ ID NO: 26 - RNA, 23 nt, synthetic construct
  source 1..23: mol_type = other RNA
agcattgcaaccgatcccaacct

SEQ ID NO: 27 - DNA, 23 bp, synthetic construct
  source 1..23: mol_type = other DNA
agcattgcaaccgatcccaacct

SEQ ID NO: 28 - DNA, 23 bp, synthetic construct
  source 1..23: mol_type = other DNA
aggattccatcggaaccgaacct

SEQ ID NO: 29 - RNA, 23 nt, synthetic construct
  source 1..23: mol_type = other RNA
caaagtgcttacagtgcaggtag

SEQ ID NO: 30 - RNA, 23 nt, synthetic construct
  source 1..23: mol_type = other RNA
ctacctgcactgtaagcactttg

SEQ ID NO: 31 - RNA, 23 nt, synthetic construct
  source 1..23: mol_type = other RNA
ctacctgcactgtaagcactttg

SEQ ID NO: 32 - RNA, 23 nt, synthetic construct
  source 1..23: mol_type = other RNA
taaagtgcttatagtgcaggtag

SEQ ID NO: 33 - RNA, 23 nt, synthetic construct
  source 1..23: mol_type = other RNA
ctacctgcactataagcacttta

SEQ ID NO: 34 - RNA, 23 nt, synthetic construct
  source 1..23: mol_type = other RNA
ctacctgcactataagcacttta

(19) United States
(12) Patent Application Publication
(10) Pub. No.: US 2025/0295603 A1
(43) Pub. Date: Sep. 25, 2025

(54) COMPOSITIONS AND METHODS FOR THE TREATMENT OF NEURODEGENERATIVE DISORDERS

(71) Applicant: Ohio State Innovation Foundation, Columbus, OH (US)

(72) Inventors: Amal AMER, New Albany, OH (US); Robert Lee, Columbus, OH (US)

(21) Appl. No.: 18/712,524

(22) PCT Filed: Nov. 22, 2022
(86) PCT No.: PCT/US2022/080364

§ 371 Date: May 22, 2024

Related U.S. Application Data
(60) Provisional application No. 63/282,056, filed on Nov. 22, 2021.

(51) Int. Cl. A61K 9/51; A61K 9/00; A61K 31/7105; A61P 25/28; C12N 5/079

(52) U.S. Cl. CPC A61K 9/5123; A61K 9/0019; A61K 31/7105; A61P 25/28; C12N 5/0622

(57) ABSTRACT

Disclosed herein compositions and methods for the treatment of neurodegenerative disorders, such as Alzheimer's disease.

### CROSS-REFERENCE TO RELATED APPLICATIONS

This application claims benefit of U.S. Provisional Application No. 63/282,056, filed Nov. 22, 2021, which is hereby incorporated herein by reference in its entirety.

### REFERENCE TO SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Jan. 31, 2025, is entitled “103361-179US1”, and is 41,801 bytes in size.

### BACKGROUND

Alzheimer's Disease (AD) is the most common form of dementia worldwide. AD is characterized by a chronic, irreversible, and progressive neuronal degradation in the human brain caused by complex pathophysiological processes. One of the main hallmarks of AD is the presence of plaques composed of insoluble, aggregated, fibrillar amyloid beta (Aβ). Aβ is a piece of the amyloid precursor protein (APP) released by neurons and exerts a protective physiological function. Once in the extracellular space, Aβ is degraded by microglia to maintain a balance between accumulation and clearance. In AD, the buildup of abnormal Aβ levels could be due to their overproduction by neuronal cells, as suggested by several reports. Otherwise, it can be due to decreased clearance of Aβ by microglia for reasons that remain unclear. The accumulation and aggregation of Aβ in the brain is a predisposing factor for AD pathobiology and usually precedes the deposition of Tau tangles. Therapies directed at removing aggregated Aβ in AD patients have failed at improving memory in clinical trials even if they reduced Aβ amounts.

These results do not refute the role of Aβ in pathogenesis of AD, they rather indicate that the deposition of Aβ in the brain provokes a damaging cascade that may not be remedied by reducing Aβ production after the damage has occurred. This strongly suggests that new approaches are needed to improve the clearance of Aβ. To do so, a better understanding of the underlying defect in Aβ clearance is necessary. Additionally, a targeted approach to deliver therapeutic cargos to specific cell types in the brains is drastically needed.

### SUMMARY

In accordance with the purposes of the disclosed materials and methods, as embodied and broadly described herein, the disclosed subject matter, in one aspect, relates to compounds, compositions and methods of making and using compounds and compositions.

In specific aspects, the disclosed subject matter relates to methods of modifying autophagy activity in microglia as well as pharmaceutical compositions for modifying autophagy activity in microglia. In further aspects, the disclosed subject matter relates to modulation of autophagy activity and compositions for modulating autophagy activity. In further aspects, the disclosed subject matter relates to methods of treating or preventing a neurodegenerative disorder (Alzheimer's disease) in a subject.

In specific embodiments, the disclosed subject matter relates to lipid particles (e.g., lipid nanoparticles) that comprise an active agent that inhibits the transcription or translation of a human miR17-92 cluster (e.g., an active agent that inhibits the transcription or translation of human miR-17). These lipid particles can include one or more lipids comprising a microglial targeting agent (e.g., one or more lipids comprising a carbohydrate moiety such as a mannose moiety) so as to target delivery of the active agent that inhibits the transcription or translation of a human miR17-92 cluster (e.g., an active agent that inhibits the transcription or translation of human miR-17) to the microglia.

In specific embodiments, the disclosed subject matter relates to lipid particles (e.g., lipid nanoparticles) that comprise a nucleic acid that hybridizes to a Mir17-92 cluster under moderate or high stringent conditions. These lipid particles can include one or more lipids comprising a microglial targeting agent (e.g., one or more lipids comprising a carbohydrate moiety such as a mannose moiety) so as to target delivery of the nucleic acid that hybridizes to a Mir17-92 cluster to the microglia. In certain embodiments, the disclosed subject matter relates to lipid particles (e.g., lipid nanoparticles) that comprise a nucleic acid hybridizes to miR-17 under moderate or high stringent conditions. These lipid particles can include one or more lipids comprising a microglial targeting agent (e.g., one or more lipids comprising a carbohydrate moiety such as a mannose moiety) so as to target delivery of the nucleic acid hybridizes to miR-17 to the microglia. Pharmaceutical compositions comprising these antagomirs and a pharmaceutically acceptable carrier are also disclosed.

Methods of using these pharmaceutical compositions to treat a patient with a neurodegenerative disorder (e.g., Alzheimer's disease), reduce the expression of human miR17-92 cluster in a subject having a neurodegenerative disorder (e.g., Alzheimer's disease), reduce the expression of miR-17 in a subject having a neurodegenerative disorder (e.g., Alzheimer's disease), and/or reducing the expression of Amyloid beta (Aβ) in a subject having a neurodegenerative disorder (e.g., Alzheimer's disease) are also disclosed. Methods of assaying levels of Mir17-92 expression are also disclosed.

### DESCRIPTION OF DRAWINGS

FIG. 1 shows the transcriptional (Panel A) and translational (Panel B) regulation of miR17-92 cluster that targets main autophagy effectors (Panel C). Pre-miRNAs are recognized and excised from pri-miRNA transcripts by the microprocessor complex, DROSHA, DGCR8, and other accessory proteins. Processed pre-miRNAs are exported to the cytoplasm via Exportin-5 where DICER, processes pre-miRNAs into 18- to 20-bp. The mature miRNA, is selectively retained into Argonaute proteins to form the miRNA-induced silencing complex (miRISC) and partially base pairs with sites typically located in the 3′UTR of mRNAs. Recognition by miRISC instigates a series of silencing activities, which include mRNA translational repression, deadenylation, decapping, and decay. As shown in Panel C, the formation of the autophagosomes requires all Atgs including Atg5, Atg7 and Nbr1.

FIGS. 2A-2D show that 5×FAD (AD) microglia degrade significantly less Aβ than WT microglia. FIG. 2A shows representative serial images of time-lapse confocal microscopy showing the degradation of Alexa-555 conjugated fibrillar Aβ in WT and 5×FAD microglia within 12 hr. As shown in FIGS. 2B and 2C, microglia underwent overnight Z-stack scanning imaging at 1 μm every 10 min for 12 hr. Quantification of Aβ showing the levels of Aβ in WT and 5×FAD microglia was performed at 1 hr to determine uptake (FIG. 2B) and after 12 hr to determine degradation (FIG. 2C). Degradation (FIG. 2C) is calculated as percent degradation in relation to the amount of internalized Aβ at 1 hr. n=13. **p<0.01. FIG. 2D is a confocal 3D reconstruction using z-stack and 3D reconstruction from Z-stack confocal images using IMARIS showing the uptake of Aβ (pseudo color purple) by microglia (green).

FIG. 3 is a plot showing that stimulation of autophagy by rapamycin in AD microglia improves Aβ degradation and inhibition of autophagy in WT microglia by 3-MA abrogates Aβ degradation. Percent degradation of Aβ within 12 hr in relation to 1 h uptake in the presence or absence of rapamycin and 3-MA. *p<0.05. n=3 Two Way Anova.

FIG. 4 is a schematic illustrates the morphology of the microglia.

FIGS. 5A-5C show that more Aβ co-localizes with LC3 and beclin1 in WT compared to 5×FAD (AD) microglia. FIG. 5A shows time lapse confocal imaging of primary microglia from WT mice expressing LC3-GFP treated with fluorescent Alexa-555 conjugated fibrillary Aβ. As shown in FIG. 5B, primary WT and AD microglia were treated with fibrillary florescent Aβ, fixed, permeabilized and labeled with fluorescent antibeclin1 antibodies. The scoring of the percent colocalization of Aβ particles with beclin1 was determined in FIG. 5C.

FIGS. 6A-6B show that autophagy effectors targeted by miR-17 are down-regulated in the brains of AD mice. FIG. 6A shows the RT PCR for Atg5, Atg7, Atg16L1 and beclin1 in AD (5×FAD) and WT mice brains. FIG. 6B shows a western blot for Atg7 from the samples described in FIG. 6A.

FIGS. 7A-7B show that autophagy effectors targeted by miR-17 are downregulated in the human brains of AD patients and Aβ accumulates compared to normal controls. FIG. 7A shows the RT PCR for Atg7 and Atg16L1 in Brodmann area 38 from the brains of human AD patients and age- and sex-matched controls FIG. 7B shows a western blot for beclin1, Atg7, P62 and Aβ1-42 from samples described in FIG. 7A. GAPDH is a loading control.

FIGS. 8A-8D show that the expression of members of the miR-17-92 cluster in the microglia (CD11b+) from the brains of 5×FAD (AD) mice is higher whereas the expression of the autophagy receptor NBR1 is lower compared to those from wild-type (WT). The expression of members of the miR-17-92 cluster determined by RT-PCR in the microglia (CD11b+) (FIG. 8A) and in the non-microglia fraction (CD11b−) (FIG. 8B) isolated from the brains of 6 month-old WT and AD mice. FIGS. 8C-8D show that the expression of the miR-17 target Nbr1 is significantly reduced in microglia rich but not in microglia poor fractions which inversely correlates with the expression of miR-17. *p<0.05, ***p<0.001.

FIG. 9 shows that microglia and Aβ plaques are closely associated in the cortex of 5×FAD mice. In Panel A, the brain of 4-month-old AD mice was sectioned and cortex was stained for microglia (Iba1, red), Aβ-plaques (green) and DAPI (blue). Extended depth projection image. Panels B and C show 3D z-stack projections of a cortex slice of the brain of 4-month-old AD stained for microglia (red, Iba1), astrocytes (green GFAP), Aβ-plaques (white), and DAPI (blue) utilizing Structured Illumination Microscopy (SIM, Nikon) resolution 20 nm.

FIGS. 10A-10B shows that the in vivo injection of naked (not enclosed in NPs) Anti-17 improves the ability of microglia to degrade Aβ. FIG. 10A shows confocal images of fluorescent-Aβ (red) at 3 and 48 h in primary microglia isolated from 5×FAD brains transfected for 48 h with either antagomir-17 (50 nM) or negative control inhibitor. Scale bar: 10 μM. FIG. 10B shows the quantification of % degradation of fluorescent Aβ (red) by degradation assay of the images represented in (A) (n=3). Values are % of Aβ volume reduction±SEM calculated by scoring 5 randomly chosen 20× fields of view. Statistical analysis was performed using paired two-tailed Student's t-test. *p<0.05, ***p<0.001.

FIGS. 11A-11B shows that Man-LNPs specifically deliver their cargo to microglia in vitro. FIG. 11A shows primary adult microglia and astrocytes untreated (NT) or treated with 150 nM antagomir-17 coupled to Alexa-555 (red) for 3 hours. Antagomir-17 was administered either inside lipid nanoparticles (LNP) with 3%-Mannose (Man-LNP), or LNPs without mannose, or conjugated to HiPerfect Transfection Reagent as a positive control, or naked antagomir-17. Cells were stained for nuclei (DAPI) (blue) and membrane stain by WGA (green) (ThermoScientific). FIG. 11B shows the quantification of results obtained in FIG. 11A.

FIG. 12 illustrates the therapeutic strategy described herein—the injection of antagomir-17 within Man-LNPs to reduce the expression of miR-17 in microglia, improve the expression of autophagy effectors and improve Aβ clearance and AD pathology.

FIGS. 13A-13D shows that intracisternal magna (ICM) injection of Anti-17 Man-LNPs into the brains of 5×FAD mice weekly for 4 weeks reduced miR-17 in microglia and reduced Aβ accumulation in the brain. FIGS. 13A and 13B show 5×FAD mice injected with either Anti-17 alone, Anti-17 in LNPs without mannose, Anti-17 within Man-LNPs, and scramble antagomir within Man-LNPs, once a week for 4 weeks starting at the age of 3.5 months. N=4 per group. Brains were homogenized and microglia (FIG. 13A) and non-microglia (FIG. 13B) fractions were separated and the expression of miR-17 was determined by RT-PCR. As shown in FIG. 13C, the same brain homogenates as in FIGS. 13A and 13B were analyzed by western blots and Aβ was identified with specific antibodies. Densitometry of Aβ band was normalized to GAPDH. In FIG. 13D, an elevated plus maze test was used for testing the mice described in FIG. 13A to test for hyperactivity. The following parameters for hyperactivity behavior testing were assessed: the distance traveled, number of open and closed arms entries, and time spent in open and closed arms. N=8 mice, 4 males, and 4 females. Only males showed significant changes. Two-way ANOVA, *p<0.05. Treatment reduced the hyperactivity in AD mice. These results support the conclusion that in vivo delivery of Anti-17 within Man-LNPs reduces the expression of miR-17 in microglia and improves the expression of its autophagy targets in microglia without affecting the expression of APP in neuronal cells.

### DETAILED DESCRIPTION

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

“Aqueous solution” refers to a composition comprising in whole, or in part, water.

“Organic lipid solution” refers to a composition comprising in whole, or in part, an organic solvent having a lipid. In some embodiments, the organic lipid solution can comprise an alkanol, most preferably ethanol. In certain embodiments, the compositions described herein can be free of organic solvents, such as ethanol.

“Lipid” refers to a group of organic compounds that are esters of fatty acids and are characterized by being insoluble in water but soluble in many organic solvents, e.g, fats, oils, waxes, phospholipids, glycolipids, and steroids.

“Amphipathic lipid” comprises a lipid in which hydrophilic characteristics derive from the presence of polar or charged groups such as carbohydrates, phosphato, carboxylic, sulfato, amino, sulfhydryl, nitro, hydroxy and other like groups, and hydrophobic characteristics can be conferred by the inclusion of a polar groups that include, but are not limited to, long chain saturated and unsaturated aliphatic hydrocarbon groups and such groups substituted by one or more aromatic, cycloaliphatic or heterocyclic group(s). Examples include phospholipids, aminolipids and sphingolipids. Phospholipids include phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Amphipathic lipids also can lack phosphorus, such as sphingolipid, glycosphingolipid families, diacylglycerols and b-acyloxyacids.

“Anionic lipid” is any lipid that is negatively charged at physiological pH, including phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

“Cationic lipid” carry a net positive charge at a selective pH, such as physiological pH, including N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N—(N′,N′-dimethylaminoethane)-carbamoyljcholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present invention. These include, for example, LIPOFECTIN®, LIPOFECT AMINE®, and TRANSFECTAM®.

As used herein, the term “isolated” means that the referenced material is removed from the environment in which it is normally found. Thus, an isolated biological material can be free of cellular components, i.e., components of the cells in which the material is found or produced. Isolated nucleic acid molecules include, for example, a PCR product, an isolated mRNA, a cDNA, or a restriction fragment. Isolated nucleic acid molecules also include, for example, sequences inserted into plasmids, cosmids, artificial chromosomes, and the like. An isolated nucleic acid molecule is preferably excised from the genome in which it may be found, and more preferably is no longer joined to non-regulatory sequences, non-coding sequences, or to other genes located upstream or downstream of the nucleic acid molecule when found within the genome. An isolated protein may be associated with other proteins or nucleic acids, or both, with which it associates in the cell, or with cellular membranes if it is a membrane-associated protein.

By “reduce” or other forms of the word, such as “reducing” or “reduction,” is meant lowering of an event or characteristic (e.g., infection). It is understood that this is typically in relation to some standard or expected value, in other words it is relative, but that it is not always necessary for the standard or relative value to be referred to. For example, “reduces infection” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as infection), diminishment of extent of infection, stabilized (i.e., not worsening) state of infection, delaying spread (e.g., infections) of the infection, delaying occurrence or recurrence of infection, delay or slowing of infection progression, and amelioration of the infected state.

The terms “patient” and “subject” preferably refers to a human in need of treatment for a neurodegenerative disease (e.g., Alzheimer's disease) and/or autophagy activity modulation. However, the term “patient” can also refer to non-human animals, preferably mammals such as dogs, cats, horses, cows, pigs, sheep and non-human primates, among others, that are in need of treatment with a composition disclosed herein.

As used herein, the terms “administering” and “administration” refer to any method of providing a pharmaceutical preparation to a patient. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In some examples, a preparation can be administered therapeutically; that is, administered to treat an existing disease or condition. In some examples, a preparation can be administered prophylactically; that is, administered for prevention of a disease or condition.

As used herein, the term “composition” is intended to encompass a product comprising the specified ingredients in the specified amounts, as well as any product which results, directly or indirectly, from combination of the specified ingredients in the specified amounts. It is also understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

A weight percent (wt. %) of a component, unless specifically stated to the contrary, is based on the total weight of the formulation or composition in which the component is included.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3rd ed., New York and London: Garland Publ., 1994, Ch. 7.

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids. Typically, hybridization of two strands at high stringency or under high stringent conditions requires that the sequences exhibit a high degree of complementarity over an extended portion of their length. Examples of high stringency conditions include: hybridization to filter-bound DNA in 0.5 M NaHPO₄, 7% SDS, 1 mM EDTA at 65° C., followed by washing in 0.1×SSC/0.1% SDS at 68° C. (where 1×SSC is 0.15M NaCl, 0.15M Na citrate) or for oligonucleotide molecules washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos)). Accordingly, the term “high stringency hybridization” refers to a combination of solvent and temperature where two strands will pair to form a “hybrid” helix only if their nucleotide sequences are almost perfectly complementary (see Molecular Biology of the Cell, Alberts et al., 3rd ed., New York and London: Garland Publ., 1994, Ch. 7).

Conditions of intermediate or moderate stringency (such as, for example, an aqueous solution of 2×SSC at 65° C.; alternatively, for example, hybridization to filter-bound DNA in 0.5 M NaHPO₄, 7% SDS, 1 mM EDTA at 65° C., and washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98: 503; Sambrook et al., Molecular Cloning: A Laboratory Manual, 2nd ed., vol. 2, ch. 9.50, CSH Laboratory Press, 1989; Ausubel et al. (eds.), 1989, Current Protocols in Molecular Biology, Vol. I, Green Publishing Associates, Inc., and John Wiley & Sons, Inc., New York, at p. 2.10.3).

As used herein, the term “standard hybridization conditions” refers to hybridization conditions that allow hybridization of sequences having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

Nucleic acid molecules that “hybridize” to any desired nucleic acids of the present invention may be of any length. In one embodiment, such nucleic acid molecules are at least 10, at least 15, at least 20, at least 30, at least 40, at least 50, and at least 70 nucleotides in length. In another embodiment, nucleic acid molecules that hybridize are of about the same length as the particular desired nucleic acid.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, GCG (Genetics Computer Group, Program Manual for the GCG Package, Version 7, Madison, Wisconsin), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc Nat Acad Sci USA 1990, 87:2264, modified as in Karlin and Altschul, Proc Natl Acad Sci USA 1993, 90:5873-5877. Such an algorithm is incorporated into the NBLAST and XBLAST programs of Altschul et al., J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al., Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

### Pharmaceutical Compositions

Provided pharmaceutical composition comprising a lipid particle encapsulating an active agent.

The active agent can comprise an agent that inhibits the transcription or translation of a human miR17-92 cluster (e.g., Mir17-19) as discussed in more detail below.

The lipid particle can comprise one or more lipids comprising a microglial targeting agent; one or more ionizable lipids, one or more cationic lipids, or a combination thereof; one or more neutral lipids; and optionally one or more PEGylated lipids.

In some embodiments, the one or more lipids comprising a microglial targeting agent are present in the lipid particle in an amount of from greater than 0 mol % to 10 mol %, such as from 0.5 mol % to 5 mol %, from 0.5 mol % to 3 mol %, or from 4 mol % to 8 mol %, based on the total components forming the lipid particle.

In some embodiments, the one or more ionizable lipids, one or more cationic lipids, or a combination thereof are present in the lipid particle in an amount of from greater than 20 mol % to 75 mol %, based on the total components forming the lipid particle.

In some embodiments, the lipid particle can comprise one or more cationic lipids. In some embodiments, the one or more cationic lipids are present in the lipid particle in an amount of from greater than 0 mol % to 10 mol %, such as from 0.5 mol % to 5 mol % or from 4 mol % to 8 mol %, based on the total components forming the lipid particle.

In some embodiments, the lipid particle can comprise one or more ionizable lipids. In some embodiments, the one or more ionizable lipids are present in the lipid particle in an amount of from 20 mol % to 65 mol % (e.g., from 30 mol % to 50 mol %), based on the total components forming the lipid particle.

In some embodiments, the one or more neutral lipids are present in the lipid particle in an amount of from 35 mol % to 80 mol % (30 mol % to 50 mol %), based on the total components forming the lipid particle.

In some embodiments, the lipid particle can comprise one or more PEGylated lipids. In some embodiments, the one or more PEGylated lipids are present in the lipid particle in an amount of from greater than 0 mol % to 5 mol % (from 0.5 mol % to 3 mol %), based on the total components forming 5 the lipid particle.

In certain embodiments, the lipid particles can comprise from greater than 0 mol % to 10 mol % of one or more lipids comprising a microglial targeting agent (e.g., one or more lipids comprising a carbohydrate moiety such as a mannose moiety); from greater than 20 mol % to 75 mol % of one or more ionizable lipids, one or more cationic lipids, or a combination thereof; from 35 mol % to 80 mol % of one or more neutral lipids; and optionally from greater than 0 mol % to 5 mol % of one or more PEGylated lipids.

The lipid particles can have an average diameter of less than 1 micron, such as from from 50 nm to 750 nm, 50 nm to 250 nm, from 50 nm to 200 nm, from 50 nm to 150 nm, or 25 from 50 nm to 100 nm. The lipid particles can have a polydispersity index (PDI) of less than 0.4.

The components of these compositions are described in more detail below.

### Active Agents

The active agent can comprise an agent that inhibits the transcription or translation of a human miR17-92 cluster (e.g., Mir17-19).

In some embodiments, the active agent can comprise a nucleic acid, such as an antagomir. Antagomirs interact with a target nucleic acid molecule (e.g., microRNA) through either canonical or non-canonical base pairing. The interaction of the antagomirs and the target molecule is designed to interrupt a processing function that normally would take place on the target molecule, such as transcription or replication. Antagomirs can be designed based on the sequence of the target nucleic acid molecule.

In the disclosed compositions, reference is made to the following sequences.

| | | SEQ |
|---|---|---|
| | | ID |
| Description | Sequence | NO.: |
| Target mature | 5′-CAA AGU GCU UAC AGU | 1 |
| miR-17-5P | GCA GGU AG-3′ | |
| sequence | | |
| Antisense | 3′-GUU UCA CGA AUG UCA | 2 |
| mature | CGU CCA UC-5′ | |
| miR-17-5P | | |
| sequence | | |
| antimiR-17 | 5′-C*+A*C*+T*G*+T*A*A* | 3 |
| | +G*+C*dA*C*+T*T*+T-3′ | |
| Mismatched | 5′-+C*+T*C*+A*G*+T*A* | 4 |
| antimiR-17 | A*+A*+G*A*T+A*T*+T-3′ | |
| (non- | | |
| targeting | | |
| control) | | |
| antimiR-17 | 5′-CAC TGT AAG CAC TTT-3′ | 5 |
| B | | |
| Mismatched | 5′-CTC AGT AAA GAT ATT-3′ | 6 |
| antimiR-17 | | |
| B | | |
| (non- | | |
| targeting | | |
| control) | | |
| AntagomiR | 5′-CT ACC TGC ACT GTA AGC | 7 |
| miR-17-5P | ACT TTG-3′ | |
| Mutant | 5′-CT ACC TCC AGT GAA AGC | 8 |
| AntagomiR | TCT ATG-3′ | |
| miR-17-5P | | |
| (Control) | | |
| Target | 5′-UAA GGU GCA UCU AGU GCA | 9 |
| mature | GAU AG-3′ | |
| miR-18A | | |
| sequence | | |
| Antisense | 3′-AUU CCA CGU AGA UCA CGU | 10 |
| mature | CUA UC-5′ | |
| miR-18A | | |
| sequence | | |
| AntagomiR | 5′-CT ATC TGC ACT AGA TGC | 11 |
| miR-18A | ACC TTA-3′ | |
| Mutant | 5′-CT ATC TCC AGT ACA TGG | 12 |
| AntagomiR | ACG TTA-3′ | |
| miR-18A | | |
| (Control) | | |
| Target | 5′-AGU UUU GCA UAG UUG CAC | 13 |
| mature | UAC A-3′ | |
| miR-19A | | |
| sequence | | |
| Antisense | 3′-U GUA GUG CAA CUA UGC | 14 |
| mature | AAA ACU-5′ | |
| miR-19A | | |
| sequence | | |
| AntagomiR | 5′-TCA AAA CGT ATC AAC GTG | 15 |
| miR-19A | ATG T-3′ | |
| Mutant | 5′-TCT AAT CGA ATG ATC GAG | 16 |
| AntagomiR | TTG T-3′ | |
| miR-19A | | |
| (Control) | | |
| Target | 5′-UAA AGU GCU UAU AGU GCA | 17 |
| mature | GGU AG-3′ | |
| miR-20 | | |
| sequence | | |
| Antisense | 3′-AUU UCA CGA AUA UCA CGU | 18 |
| mature | CCA UC-5′ | |
| miR-20 | | |
| sequence | | |
| AntagomiR | 5′-CT ACC TGC ACT ATA AGC | 19 |
| miR-20 | ACT TTA-3′ | |
| Mutant | 5′-CT TCC AGC AGT AAA ACC | 20 |
| AntagomiR | AGT ATA-3′ | |
| miR-19A | | |
| (Control) | | |
| Target | 5′-AGU UUU GCA GGU UUG CAU | 21 |
| mature | CCA GC-3′ | |
| miR-19B-1 | | |
| sequence | | |
| Antisense | 3′-UCA AAA CGU CCA AAG GUA | 22 |
| mature | GGU CG-5′ | |
| miR-19B-1 | | |
| sequence | | |
| AntagomiR | 5′-GC TGG ATG GAA ACC TGC | 23 |
| miR-19B-1 | AAA ACT-3′ | |
| Mutant | 5′-GC AGG AAG CAA AGC TCC | 24 |
| AntagomiR | TAA ACT-3′ | |
| miR-19B-1 | | |
| (Control) | | |
| Target | 5′-AGG UUG GGA UCG GUU GCA | 25 |
| mature | AUG CU-3′ | |
| miR-92A-1 | | |
| sequence | | |
| Antisense | 3′-UCC AAC CCU AGC CAA CGU | 26 |
| mature | UAC GA-5′ | |
| miR-92A-1 | | |
| sequence | | |
| AntagomiR | 5′-AG CAT TGC AAC CGA TCC | 27 |
| miR-92A-1 | CAA CCT-3′ | |
| Mutant | 5′-AG GAT TCC ATC GGA ACC | 28 |
| AntagomiR | GAA CCT-3′ | |
| miR-92A-1 | | |
| (Control) | | |
| Target | 5′-CAA AGU GCU UAC AGU GCA | 29 |
| mature | GGU AG-3′ | |
| miR-17-5P | | |
| sequence | | |
| (mouse) | | |
| Antisense | 3′-GUU UCA CGA AUG UCA CGU | 30 |
| mature | CCA UC-5′ | |
| miR-17-5P | | |
| sequence | | |
| (mouse) | | |
| AntagomiR | 5′-CU ACC UGC ACU GUA AGC | 31 |
| miR-17-5P | ACU UUG-3′ | |
| (mouse) | | |
| Target | 5′-UAA AGU GCU UAU AGU GCA | 32 |
| mature | GGU AG-3′ | |
| miR-20 | | |
| sequence | | |
| (mouse) | | |
| Antisense | 3′-AUU UCA CGA AUA UCA CGU | 33 |
| mature | CCA UC-5′ | |
| miR-20 | | |
| sequence | | |
| (mouse) | | |
| AntagomiR | 5′-CU-ACC UGC ACU AUA AGC | 34 |
| miR-20 | ACU UUA-3′ | |
| (mouse) | | |

In some embodiments, the nucleic acid can include one or more locked nucleic acid bases (e.g., Affinity Plus™ locked nucleic acid bases) to increase nuclease stability and affinity (Tₘ) of the oligonucleotide to the target mRNA. The presence of a locked nucleic acid base (e.g., an Affinity Plus™ LNA nucleotide) is indicated by a plus (+) before the base.

In some embodiments, the nucleic acid can include phosphorothioate (PS) modifications to increase nuclease resistance. In the sequences above, the presence of a phosphorothioate internucleoside linkage is indicated with an asterisk (*).

The nucleic acid can comprise an antisense oligonucleotide (ASO). ASOs are DNA oligos, typically 15-22 bases long designed in antisense orientation to the RNA of interest. Hybridization of the ASO to the target RNA can trigger RNase H cleavage of the RNA, which can inhibit the function of non-coding RNAs (e.g., miRNAs, siRNAs, piRNAs, snoRNAs, snRNAs, exRNAs, scaRNAs and lncRNAs) or prevent protein translation of mRNAs.

In specific examples, disclosed herein are pharmaceutical composition comprising an antagomir. In the disclosed compositions the antagomir comprises, or is, a nucleic acid that hybridizes to a Mir17-92 cluster under moderate stringent conditions. The nucleic acid can be a non-naturally occurring nucleic acid. In other examples, the nucleic acid hybridizes to the Mir17-92 cluster under high stringent conditions. Reference to Mir17-92 includes any part of the cluster. The Mir17-92 cluster can be from a mouse or human, preferably a human. In specific examples, however, the nucleic acid can hybridize to Mir17, Mir18a, Mir19a, Mir20a, Mir19b, or Mir92 under moderate or under high stringent conditions. In specific examples, the nucleic acid hybridizes to Mir17 under moderate or under high stringent conditions. In further examples, the composition can comprise more than one antagomir, each antagomir comprising a nucleic acid that hybridizes under moderate or stringent conditions to a different part of the Mir17-92 cluster.

In some embodiments, the nucleic acid hybridizes to SEQ ID Nos.: 1, 9, 13, 17, 21, or 25. In certain embodiments, the nucleic acid hybridizes to SEQ ID Nos.: 1.

In some embodiments, the nucleic acid has at least 80%, at least 90%, at least 95%, or at least 99% sequence homology to SEQ ID Nos: 3, 5, 7, 11, 15, 19, 23, or 27. In some embodiments, the nucleic acid has at least 80%, at least 90%, at least 95%, or at least 99% sequence homology to SEQ ID Nos: 3, 5, or 7. In some embodiments, the nucleic acid has at least 80%, at least 90%, at least 95%, or at least 99% sequence homology to SEQ ID Nos: 3 or 5. In some embodiments, the nucleic acid has at least 80%, at least 90%, at least 95%, or at least 99% sequence homology to SEQ ID No: 3. For example, the nucleic acid can have from 1 to 7, from 1 to 6, from 1 to 5, from 1 to 4, from 1 to 3, from 1 to 2, substitutions, e.g., 1, 2, 3, 4, 5, 6, or 7 substitutions.

In some embodiments, the nucleic acid can have SEQ ID Nos: 3, 5, 7, 11, 15, 19, 23, or 27. In some embodiments, the nucleic acid can have SEQ ID Nos: 3, 5, or 7. In some embodiments, the nucleic acid can have SEQ ID Nos: 3 or 5. In some embodiments, the nucleic acid can have SEQ ID No: 3.

In the disclosed compositions, the composition can comprise more than one antagomir, each antagomir comprising a nucleic acid that hybridizes to a different part of the Mir17-92 cluster under moderate stringent conditions. For example, the composition can comprise multiple nucleic acids that hybridize to difference sequences chosen from SEQ ID NO.: 1, 9, 13, 17, 21, or 25 under moderate or high stringent conditions. In other examples, any two or more of the nucleic acids disclosed herein can be combined in the disclosed compositions.

The disclosed nucleic acids can also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The disclosed nucleic acids can have from 15 to 30 nucleosides, e.g., from 18 to 27, from 21 to 24, from 17 to 28, from 19 to 27, from 21 to 25, from 16 to 29, from 17 to 28, from 18 to 28, from 19 to 27, from 20 to 26, from 21 to 25, from 22 to 24 nucleosides.

The disclosed antagomirs can be obtained commercially, or can be prepared by nucleic acid synthetic techniques known to those of skill in the art. For example, they can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, MA or ABI Model 380B). Synthetic methods useful for making oligonucleotides are also described by Ikuta et al., Ann. Rev. Biochem. 1984; 53:323-356, (phosphotriester and phosphite-triester methods), and Narang et al., Methods Enzymol. 1980; 65:610-620, (phosphotriester method).

### Lipids Comprising a Microglial Targeting Agent

As described above, the lipid particles described herein can comprise one or more lipids comprising a microglial targeting agent.

Lipids comprising a microglial targeting agent can include a microglial targeting agent, such as an agent that binds to microglial mannose receptors. In some embodiments, the microglial targeting agent can comprise a carbohydrate, such as a monosaccharide (e.g., mannose, galactose, or a derivative thereof. In certain embodiments, the microglial targeting agent can comprise mannose or a derivative thereof.

As used herein, a “derivative” of a compound is a compound structurally similar to the compound of which it is a derivative. Many derivatives are functional derivatives. That is, the derivatives generally a desired function similar to the compound to which it is a derivative. By way of example, mannose is described herein as a microglial targeting agent because mannose binds microglial mannose receptors. Accordingly, a functional mannose derivative is a mannose derivative that may bind a microglia mannose receptor with the same or similar affinity as mannose (e.g., has dissociation constant that is within about a 100 fold range of that of mannose, such as within about a 10 fold range of that of mannose).

The microglial targeting agent can be covalently bound to any of the lipids described herein to afford a lipid comprising a microglial targeting agent. In certain embodiments, the microglial targeting agent can be covalently linked to a lipid, such as distearoyl-snglycero-3-phosphoethanolamine (DSPE), by a hydrophilic polymer, such as polyethylene glycol (PEG).

By way of example, DSPE-PEG-mannose may be synthesized through amide coupling of D-mannosamine hydrochloride with DSPE-PEG-COOH; e.g., as shown in the following reaction scheme:

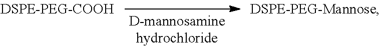

where EDC is 1-ethyl-3-(3-(dimethylamino)-propyl)carbodiimide.

In some embodiments, the one or more lipids comprising a microglial targeting agent can comprise greater than 0 mol % (e.g., at least 0.5 mol %, at least 1 mol %, at least 1.5 mol %, at least 2 mol %, at least 2.5 mol %, at least 3 mol %, at least 3.5 mol %, at least 4 mol %, at least 4.5 mol %, at least 5 mol %, at least 5.5 mol %, at least 6 mol %, at least 6.5 mol %, at least 7 mol %, at least 7.5 mol %, at least 8 mol %, at least 8.5 mol %, at least 9 mol %, or at least 9.5 mol %) of the total components forming the lipid particle. In some embodiments, the one or more PEGylated lipids comprise 10 mol % or less (e.g., 9.5 mol % or less, 9 mol % or less, 8.5 mol % or less, 8 mol % or less, 7.5 mol % or less, 7 mol % or less, 6.5 mol % or less, 6 mol % or less, 5.5 mol % or less, 5 mol % or less, 4.5 mol % or less, 4 mol % or less, 3.5 mol % or less, 3 mol % or less, 2.5 mol % or less, 2 mol % or less, 1.5 mol % or less, 1 mol % or less, or 0.5 mol % or less) of the total components forming the lipid particle

The one or more PEGylated lipids are present in the lipid particle in an amount ranging from any of the minimum values described above to any of the maximum values described above. For example, in some embodiments, the one or more PEGylated lipids are present in the lipid particle in an amount of from greater than 0 mol % to 10 mol % (e.g., from 0.5 mol % to 10 mol %, from 0.5 mol % to 8 mol %, from 0.5 mol % to 3.5 mol %, from 4 mol % to 8 mol %, from 0.5 mol % to 3 mol %, from 3 mol % to 6 mol %, or from 6 mol % to 10 mol %) of the total components forming the lipid particle.

### Ionizable Lipids

As described above, the lipid particles described herein can comprise one or more ionizable lipids. An “ionizable lipid” is a lipid that carries a charge that is pH-dependent. The one or more ionizable lipids in the composition described herein can comprise ionizable cationic lipids which carry a positive or neutral carge depending on pH.

Generally, in lipid-based formulations for nucleic acid delivery, either a cationic lipid or an ionizable lipid is used to enable electrostatic interaction with the negatively charged cargo. A cationic lipid is typically defined as a lipid that carries a permanent positive charge(s) that typically comes from a quaternary amine. Examples of a cationic lipids include DOTAP, DOTMA, DDAB, and DODAC. In contrast, ionizable lipids include a chemical moiety, such as a tertiary amine(s), which is positively charged at acidic pH but becomes uncharged at neutral to basic pH. Ionizable lipids can have a pKa value in a biologically relevant range. However, the pKa value of such a lipid is highly dependent on the method used to measure it, resulting in up to 3 units of difference in numerical values for the same lipid. This has been documented in a recent article by Carrasco et al. Communications Biology volume 4, Article number: 956 (2021).

Examples of ionizable lipids are DODMA (N,N-dimethyl-2,3-dioleyloxypropylamine), DODAP, DLinDMA (1,2-dilinoleyloxy-3-dimethylaminopropane), DLinMC3DMA (dilinoleylmethyl-4-dimethylaminobutyrate), DLinKC2DMA (2-dilinoleyl-4-dimethylaminoethyl-[1,3]-dioxolane), ALC-0315 ([(4-hydroxybutyl)azanediyl]di(hexane-6,1-diyl)bis(2-hexyldecanoate)), SM-102 (9-heptadecanyl 8-{(2-hydroxyethyl)[6-oxo-6-(undecyloxy)hexyl]amino}octanoate), Merck-32 (see e.g., WO 2012/018754), Acuitas-5 (see e.g., WO 2015/199952), KL-10 (see e.g., U.S. Patent Application Publication 2012/0295832), C12-200 (see e.g., Love, K T et al., PNAS, 107: 1864 (2009)), and the like. Ionizable lipids also include those disclosed in U.S. Pat. Nos. 8,158,601, 9,593,077, 9,365,610, 9,567,296, 9,580,711, and 9,670,152, International Publication Nos. WO 2012/018754, WO 2015/199952, WO 2019/191780, and U.S. Patent Application Publication Nos. 2012/0295832, 2017/0190661 and 2017/0114010, each of which is incorporated herein by reference in its entirety.

In some embodiments, the one or more ionizable lipids can comprise a lipid headgroup comprising a tertiary amine. In certain embodiments, the one or more ionizable lipids can comprise N,N-dimethyl-2,3-dioleyloxypropylamine (DODMA), [(4-hydroxybutyl)azanediyl]di(hexane-6,1-diyl)bis(2-hexyldecanoate) (ALC-0315); 9-heptadecanyl 8-{(2-hydroxyethyl)[6-oxo-6-(undecyloxy)hexyl]amino}octanoate (SM-102), MC-3; KC-2; or any combination thereof.

In some embodiments, the one or more ionizable lipids comprise at least 20 mol % (e.g., at least 25 mol %, at least 30 mol %, at least 35 mol %, at least 40 mol %, at least 45 mol %, at least 50 mol %, at least 55 mol %, or at least 60 mol %) of the total components forming the lipid particle. In some embodiments, the one or more ionizable lipids comprise 65 mol % or less (e.g., 60 mol % or less, 55 mol % or less, 50 mol % or less, 45 mol % or less, 40 mol % or less, 35 mol % or less, 30 mol % or less, or 25 mol % or less) of the total components forming the lipid particle

The one or more ionizable lipids can be present in the lipid particle in an amount ranging from any of the minimum values described above to any of the maximum values described above. For example, in some embodiments, the one or more ionizable lipids are present in the lipid particle in an amount of from 20 mol % to 65 mol % (e.g., from 30 mol % to 50 mol %) of the total components forming the lipid particle.

### Neutral Lipids

As described above, the lipid particles described herein can comprise one or more neutral lipids.

Examples of neutral lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having Cio-C24 carbon chains, e.g., lauroyl, myristoyl, palmitoyl, stearoyl, or oleoyl.

Additional examples of neutral lipids include sterols such as cholesterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In preferred embodiments, the cholesterol derivative is a polar analogue such as cholesteryl-(4′-hydroxy)-butyl ether. Other examples of neutral lipids include nonphosphorous containing lipids such as, e.g, stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the one or more neutral lipids can comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

In some embodiments, the one or more neutral lipids comprise at least 35 mol % (e.g., at least 40 mol %, at least 45 mol %, at least 50 mol %, at least 55 mol %, at least 60 mol %, at least 65 mol %, at least 70 mol %, or at least 75 mol %) of the total components forming the lipid particle. In some embodiments, the one or more neutral lipids comprise 80 mol % or less (e.g., 75 mol % or less, 70 mol % or less, 65 mol % or less, 60 mol % or less, 55 mol % or less, 50 mol % or less, 45 mol % or less, or 40 mol % or less) of the total components forming the lipid particle

The one or more neutral lipids can be present in the lipid particle in an amount ranging from any of the minimum values described above to any of the maximum values described above. For example, in some embodiments, the one or more neutral lipids are present in the lipid particle in an amount of from 35 mol % to 80 mol % (30 mol % to 50 mol %) of the total components forming the lipid particle.

### PEGylated Lipids

As described above, the compositions described herein can optionally comprise one or more PEGylated lipids. The one or more PEGylated lipids are useful in that they can reduce or prevent the aggregation of lipid particles.

PEG is a linear, water-soluble polymer of ethylene PEG repeating units with two terminal hydroxyl groups. PEGs are classified by their molecular weights; and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NEh), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH2).

Examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to glycerides forming a glycol, e.g., 1,2-dimyristoyl-sn-glycerol, methoxy-PEG glycol (PEG-DMG), PEG conjugated to ceramides, PEG conjugated to cholesterol, or a derivative thereof, and mixtures thereof. In some examples, the one or more PEGylated lipids can comprise, for example, a PEG-ditetradecylacetamide, a PEG-myristoyl diglyceride, a PEG-diacylglycerol, a PEG dialkyloxypropyl, a PEG-phospholipid, a PEG-ceramide, or any combinations thereof.

The PEG moiety of the PEG-lipid conjugates described herein may comprise an average molecular weight ranging from 550 Daltons to 10,000 Daltons. In certain instances, the PEG moiety has an average molecular weight of from 750 Daltons to 5,000 Daltons (e.g, from 1,000 Daltons to 5,000 Daltons, from 1,500 Daltons to 3,000 Daltons, from 750 Daltons to 3,000 Daltons, from 750 Daltons to 2,000 Daltons). In some embodiments, the PEG moiety has an average molecular weight of 2,000 Daltons or 750 Daltons.

In certain instances, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one embodiment, the linker moiety is a non-ester-containing linker moiety. Suitable non-ester-containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (-0-), succinyl (-(0)CCH2CH2C(0)-), succinamidyl (—NHC(0)CH2CH2C(0)NH—), ether, disulphide, as well as combinations thereof (such as a linker containing both a carbamate linker moiety and an amido linker moiety). In some embodiments, a carbamate linker is used to couple the PEG to the lipid. In other embodiments, an ester-containing linker moiety can be used to couple the PEG to the lipid. Suitable ester-containing linker moieties include, e.g., carbonate (—OC(O)O—), succinoyl, phosphate esters (—O—(O)POH—O—), sulfonate esters, and combinations thereof.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (Ci₄), palmitoyl (Ci6), stearoyl (Cis), and icosoyl (C20). In preferred embodiments, R¹ and R² are the same, i.e., R¹ and R² are both myristoyl (i.e., dimyristoyl), R¹ and R² are both stearoyl (i.e., distearoyl).

The term “dialkyloxyalkyl” or “DAA” includes a compound having 2 alkyl chains, R and R′, both of which have independently between 2 and 30 carbons. The alkyl groups can be saturated or have varying degrees of unsaturation.

Examples of PEG-DAA conjugates include PEG-didecyloxypropyl (C10), a PEG-dilauryloxypropyl (C12), a PEG-dimyristyloxypropyl (C14), a PEG-dipalmityloxypropyl (C16), and PEG-distearyloxypropyl (C18). In some of these embodiments, the PEG can have an average molecular weight of 750 or 2,000 Daltons. In certain embodiments, the terminal hydroxyl group of the PEG can be substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In some embodiments, the one or more PEGylated lipids comprise greater than 0 mol % (e.g., at least 0.5 mol %, at least 1 mol %, at least 1.5 mol %, at least 2 mol %, at least 2.5 mol %, at least 3 mol %, at least 3.5 mol %, at least 4 mol %, or at least 4.5 mol %) of the total components forming the lipid particle. In some embodiments, the one or more PEGylated lipids comprise 5 mol % or less (e.g., 4.5 mol % or less, 4 mol % or less, 3.5 mol % or less, 3 mol % or less, 2.5 mol % or less, 2 mol % or less, 1.5 mol % or less, 1 mol % or less, or 0.5 mol % or less) of the total components forming the lipid particle

The one or more PEGylated lipids are present in the lipid particle in an amount ranging from any of the minimum values described above to any of the maximum values described above. For example, in some embodiments, the one or more PEGylated lipids are present in the lipid particle in an amount of from greater than 0 mol % to 5 mol % of the total components forming the lipid particle.

### Cationic Lipids

As described above, the lipid blend described herein can comprise one or more cationic lipids (e.g., lipids bearing a quaternary ammonium moiety). Examples of cationic lipids include, for example, DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA (2,3-dioleyloxy-N-[2-(spermine carboxamido)ethyl]-N,N-dimethyl-1-propanaminium trifluoroacetate), DORIE (N-[1-(2,3-dioleyloxypropyl)]-N,N-dimethyl-N-hydroxyethylammonium bromide), DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammonium, oligofectamine, lipids described in U.S. Pat. No. 5,049,386, N-[1-(2,3-dioleyloxypropyl)]-N,N-dimethyl-N-hydroxyethylammonium bromide (DORIE), 2,3-dioleyloxy-N-[2-(spermine carboxamido)ethyl]-N,N-dimethyl-1-propanaminium trifluoroacetate (DOSPA), and the like as disclosed in International Publication Nos. WO91/16024 and WO97/019675; DLinDMA and the like as disclosed in International Publication No. WO2005/121348; and DLin-K-DMA and the like as disclosed in International Publication No. WO2009/086558; and (3R,4R)-3,4-bis((Z)-Hexadec-9-enyloxy)-1-methylpyrrolidine, and N-Methyl-N,N-bis(2-((Z)-octadec-6-enyloxy)ethyl)amine and the like as disclosed in International Publication No. WO2011/13636.

In some embodiments, the one or more PEGylated lipids comprise greater than 0 mol % (e.g., at least 0.5 mol %, at least 1 mol %, at least 1.5 mol %, at least 2 mol %, at least 2.5 mol %, at least 3 mol %, at least 3.5 mol %, at least 4 mol %, at least 4.5 mol %, at least 5 mol %, at least 5.5 mol %, at least 6 mol %, at least 6.5 mol %, at least 7 mol %, at least 7.5 mol %, at least 8 mol %, at least 8.5 mol %, at least 9 mol %, or at least 9.5 mol %) of the total components forming the lipid particle. In some embodiments, the one or more PEGylated lipids comprise 10 mol % or less (e.g., 9.5 mol % or less, 9 mol % or less, 8.5 mol % or less, 8 mol % or less, 7.5 mol % or less, 7 mol % or less, 6.5 mol % or less, 6 mol % or less, 5.5 mol % or less, 5 mol % or less, 4.5 mol % or less, 4 mol % or less, 3.5 mol % or less, 3 mol % or less, 2.5 mol % or less, 2 mol % or less, 1.5 mol % or less, 1 mol % or less, or 0.5 mol % or less) of the total components forming the lipid particle

The one or more PEGylated lipids are present in the lipid particle in an amount ranging from any of the minimum values described above to any of the maximum values described above. For example, in some embodiments, the one or more PEGylated lipids are present in the lipid particle in an amount of from greater than 0 mol % to 10 mol % (e.g., from 0.5 mol % to 10 mol %, from 0.5 mol % to 8 mol %, from 0.5 mol % to 3.5 mol %, from 4 mol % to 8 mol %, from 0.5 mol % to 3 mol %, from 3 mol % to 6 mol %, or from 6 mol % to 10 mol %) of the total components forming the lipid particle.

### Additional Components

The composition described herein can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, (e.g., intrathecal, intracisternal, or intraventricular, administration). Administration can be in the form of a single bolus dose, or may be, for example, by a continuous perfusion pump. In some embodiments, the compositions provided herein are suitable for intravenous administration. In some embodiments, the compounds provided herein are suitable for intracranial administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

Also provided are pharmaceutical compositions which contain, as the active ingredient, a compound provided herein in combination with one or more pharmaceutically acceptable carriers (e.g., excipients). In making the pharmaceutical compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be, for example, in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The active compound can be effective over a wide dosage range and is generally administered in an effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The compositions provided herein can be administered one from one or more times per day to one or more times per week; including once every other day. The skilled artisan will appreciate that certain factors can influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a compound described herein can include a single treatment or a series of treatments.

Dosage, toxicity and therapeutic efficacy of the compounds provided herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds exhibiting high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

### Methods of Use

The compositions described herein can be used to deliver one or more active agents to cells (e.g., in vivo, ex vivo, or in vitro). In certain embodiments, the compositions described herein can be used to deliver one or more active agents to a microglia cell (e.g., in vivo, ex vivo, or in vitro). Accordingly, provided herein are method of delivering an active agent to a cell (e.g., in vivo, ex vivo, or in vitro), such as a microglia cell, that comprise contacting the cell with a composition described herein. Also provided are methods for in vivo delivery of an active agent to a cell, said method comprising administering to a mammalian subject (e.g., a human) a composition described herein. In some embodiments, the administration can comprise systemic administration (e.g., intravenous injection or infusion). In some embodiments, the administration can comprise local administration to the brain.

In some embodiments, the compositions described herein can be used to modify or modulate autophagy activity in microglia. In further aspects, the disclosed subject matter relates to methods of treating or preventing a neurodegenerative disorder (Alzheimer's disease) in a subject.

In specific embodiments, the compositions described herein can be used to inhibit the transcription or translation of a human miR17-92 cluster (e.g., an active agent that inhibits the transcription or translation of human miR-17). These compositions can target delivery of the active agent that inhibits the transcription or translation of a human miR17-92 cluster (e.g., an active agent that inhibits the transcription or translation of human miR-17) to the microglia.

In some embodiments, the compositions described herein can be administered to a subject in need thereof to treat or prevent a neurodegenerative disease or neurodegenerative disorder. The terms “neurodegenerative disease” and “neurodegenerative disorder” are used interchangeably herein.

Neurodegenerative diseases are a class of neurological diseases that are characterized by the progressive loss of the structure and function of neurons and neuronal cell death. Inflammation has been implicated for a role in several neurodegenerative diseases. Progressive loss of motor and sensory neurons and the ability of the mind to refer sensory information to an external object is affected in different kinds of neurodegenerative diseases. Non-limiting examples of neurodegenerative diseases include ALS, e.g., familial ALS or sporadic ALS, Parkinson's Disease, Alzheimer's Disease, epilepsy, or pain.

A health care professional may diagnose a subject as having a neurodegenerative disease by the assessment of one or more symptoms of a neurodegenerative disease in the subject. Non-limiting symptoms of a neurodegenerative disease in a subject include difficulty lifting the front part of the foot and toes; weakness in arms, legs, feet, or ankles; hand weakness or clumsiness; slurring of speech; difficulty swallowing; muscle cramps; twitching in arms, shoulders, and tongue; difficulty chewing; difficulty breathing; muscle paralysis; partial or complete loss of vision; double vision; tingling or pain in parts of body; electric shock sensations that occur with head movements; tremor; unsteady gait; fatigue; dizziness; loss of memory; disorientation; misinterpretation of spatial relationships; difficulty reading or writing; difficulty concentrating and thinking; difficulty making judgments and decisions; difficulty planning and performing familiar tasks; depression; anxiety; social withdrawal; mood swings; irritability; aggressiveness; changes in sleeping habits; wandering; dementia; loss of automatic movements; impaired posture and balance; rigid muscles; bradykinesia; slow or abnormal eye movements; involuntary jerking or writhing movements (chorea); involuntary, sustained contracture of muscles (dystonia); lack of flexibility; lack of impulse control; and changes in appetite. A health care professional may also base a diagnosis, in part, on the subject's family history of a neurodegenerative disease. A health care professional may diagnose a subject as having a neurodegenerative disease upon presentation of a subject to a health care facility (e.g., a clinic or a hospital). In some instances, a health care professional may diagnose a subject as having a neurodegenerative disease while the subject is admitted in an assisted care facility. Typically, a physician diagnoses a neurodegenerative disease in a subject after the presentation of one or more symptoms.

The pharmaceutical compositions described herein can be administered to a subject in need thereof to treat or prevent a neurodegenerative disorder (e.g., Alzheimer's disease), reduce the expression of human miR17-92 cluster in a subject having a neurodegenerative disorder (e.g., Alzheimer's disease), reduce the expression of miR-17 in a subject having a neurodegenerative disorder (e.g., Alzheimer's disease), and/or reducing the expression of Amyloid beta (Aβ) in a subject having a neurodegenerative disorder (e.g., Alzheimer's disease) are also disclosed.

These methods can comprise administering an effective amount of a composition described herein. In some embodiments, the administration can comprise local administration to the brain. As used herein, the phrase “effective amount” refers to the amount of active compound or pharmaceutical agent that elicits the biological or medicinal response that is being sought in a tissue, system, animal, individual or human by a researcher, veterinarian, medical doctor or other clinician. An effective amount of a compound provided herein can range, for example, from about 0.01 mg/kg to about 1000 mg/kg, (e.g., from about 0.1 mg/kg to about 100 mg/kg, from about 1 mg/kg to about 100 mg/kg). Effective doses will also vary depending on route of administration, as well as the possibility of co-usage with other agents.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. Targeting Microglia to Alleviate Alzheimer's Disease Pathobiology

### SUMMARY

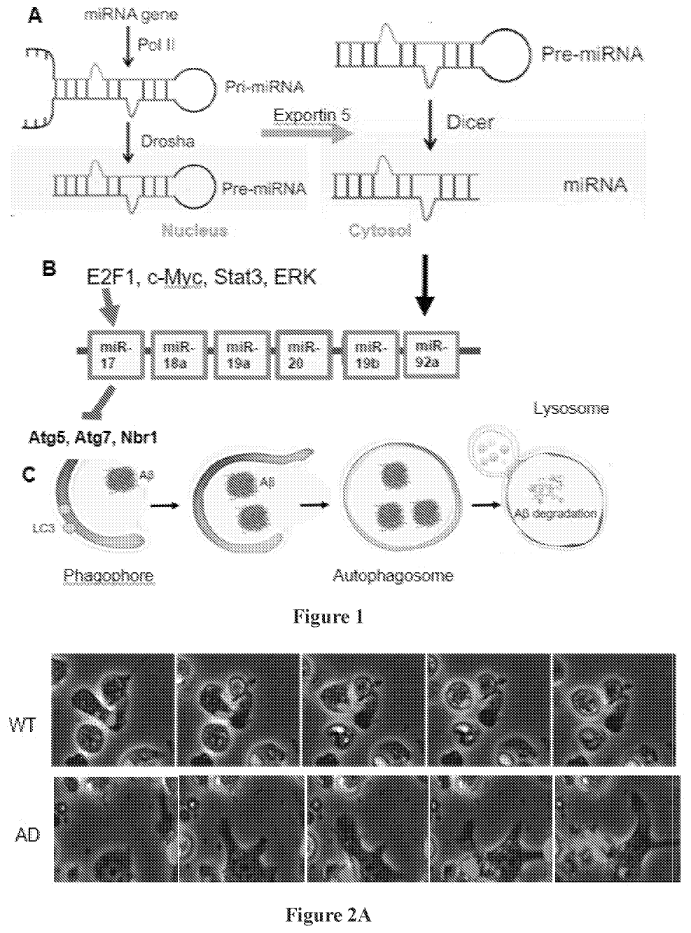

Microglia are the brain's resident immune cells that maintain tissue homeostasis. Under normal conditions, amyloid (Aβ) is released extracellularly by neurons as a neuroprotective molecule while healthy microglia ingest and degrade Aβ to maintain a balance between production and clearance. One of the hallmarks of Alzheimer's Disease (AD) is the presence of plaques composed of aggregated, fibrillar amyloid beta (Aβ) that usually precedes the deposition of Tau. As described below, Aβ can be degraded within microglia largely by autophagy, which is a conserved degradative process within eukaryotic cells that can degrade phagocytosed particles and protein aggregates (FIG. 1). Brain sections from AD patients and AD mouse models are characterized by a significant accumulation of aggregated Aβ in the cortex compared to their age- and sex matched controls. It is still unclear why microglia fail to clear Aβ in the AD brain. However, data shows that (i) microglia from the 5×FAD (AD) mouse model congregate in the areas where Aβ plaques accumulate; (ii) isolated primary microglia from adult AD mouse display defective Aβ degradation in comparison to wild-type (WT) microglia; (iii) treatment of primary AD microglia with the autophagy stimulator rapamycin improves the degradation of fibrillar Aβ, whereas inhibition of autophagy in primary healthy WT microglia with 3MA reduces fibrillar Aβ degradation; (iv) most vacuoles containing Aβ exhibit the autophagy markers LC3 and beclin1 in primary WT microglia but not in AD microglia; (v) the expression of autophagy effectors Nbr1, Atg5, Atg7 and beclin1 are significantly reduced in isolated adult AD mouse microglia (CD11b+) and not in CD11b− fraction which contains astrocytes and neuronal cells; (vi) concurrently, human and mouse microglia in the AD brains express significantly high levels of miR-17-92 cluster which is known to target autophagy effectors. While some have suggested that autophagy may be altered in neurons in Alzheimer's disease (AD), the status of autophagy in the immune cell of the brain, i.e. microglia is still enigmatic. To confirm that the isolated adult microglia reflect similar phenotype to those still within the brain, using in situ hybridization, (vii) we found that the expression of miR-17-92 cluster in human and mouse AD brain sections is high in microglia adjacent to Aβ plaques while undetectable in other cells and in areas away from Aβ plaques. This is the first report of a significant increase of miR-17-92 cluster in the microglia of AD humans and adult mice with accompanying reduction of its autophagy targets. Importantly, (viii) miR-17 was significantly increased in both mouse and human microglia and (ix) reducing its expression in AD microglia in vitro, was sufficient to significantly improve their ability to degrade Aβ. Finally, (xi) the injection of live AD mice with antagomir to miR-17 (Anti-17) within mannose-labeled nanoparticles (Man-LNPs) which specifically target microglia, significantly and specifically reduced the elevated expression of miR-17 in microglia and reduced the expression of Aβ in the brain. Together, these results demonstrate that elevated miR-17 in the microglia of the AD brain is responsible for the down-regulation of autophagy effectors, which leads to reduced degradation of Aβ by microglia (FIG. 1).

MiR-17 expression is elevated in microglia adjacent to Aβ plaques in human AD brain sections. To determine the mechanism of upregulation of the miR-17, we will determine the effect of Aβ and/or Tau exposure on the expression of miR-17; identify the factors that control transcription of miR-17-92 cluster; and identify the 3′UTR region of autophagy effectors bound by miR-17 and the direct consequence of this binding on autophagy transcript and protein expression (FIG. 1).

Data shows that delivery of Anti-17 Man-LNPs to live AD mice reduced miR-17 in microglia and reduced Aβ levels in their brains. Yet, the underlying mechanism and downstream functional consequences of reducing the expression of miR-17 on AD pathology are still unknown. Thus, we will determine the effect of Anti-17 Man-LNPs injection on the expression of miR-17 autophagy targets; determine if injection of Anti-17 Man-LNPs in vivo reduces microglia activation and neuronal loss; and determine if injection of Anti-17 Man-LNPs in vivo prevents or delays the deterioration in memory of AD mice.

Live AD mice will also be injected with Anti-17 Man-LNPs then microglia will be isolated to test their phenotypes ex vivo. This will allow us to determine if the Aβ-containing vacuoles acquire autophagy markers and mature into autophago-lysosomes; determine if isolated microglia regained their ability to degrade fibrillar Aβ in functional lysosomes; and characterize the phenotypes of isolated microglia using single cell RNAseq analysis.

### INTRODUCTION

Autophagy is a conserved cellular process for the degradation of extracellular and intracellular unwanted materials (cargo). The cargo is targeted by autophagy when marked by specific molecules such as poly ubiquitin and in rare cases Galactin-826. Then, autophagy receptors such as p6227, NDP5228, OPTN (optineurin), and NBR1 (neighbor of BRCA1 gene 1) bind ubiquitin and recruit LC3 and the nascent forming autophagosome. LC3 is an essential autophagy effector involved in the formation of autophagosomes and is often used as an autophagy marker. Autophagy can be stimulated by starvation, stress, or pharmaceutical compounds such as rapamycin, and inhibited by 3-methyl adenine (3-MA). Typically, when extracellular materials destined for autophagy are phagocytosed by immune cells such as macrophages and microglia, they are enclosed within autophagosomes, which mature and traffic to fuse with acidic lysosomes where they get degraded (FIG. 1).

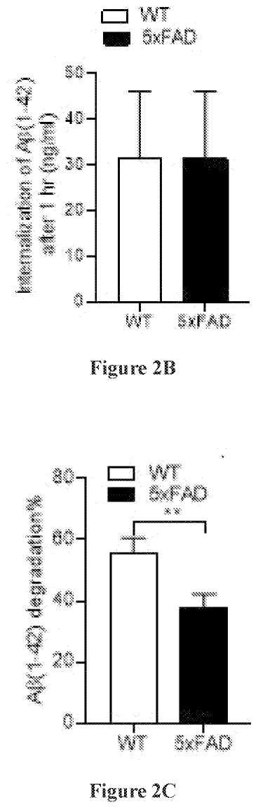

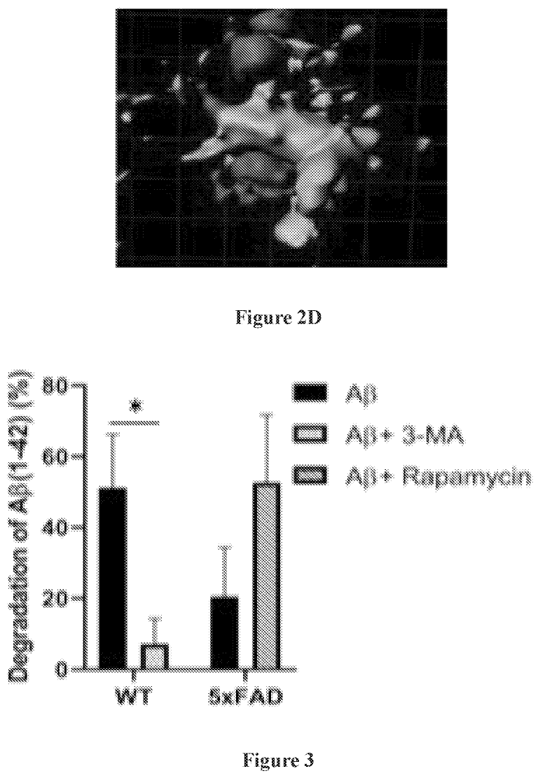

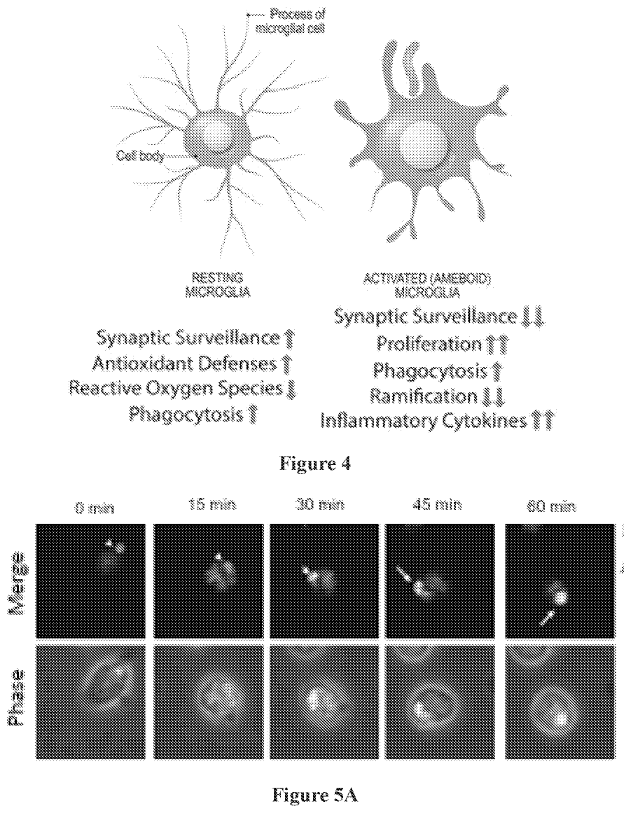

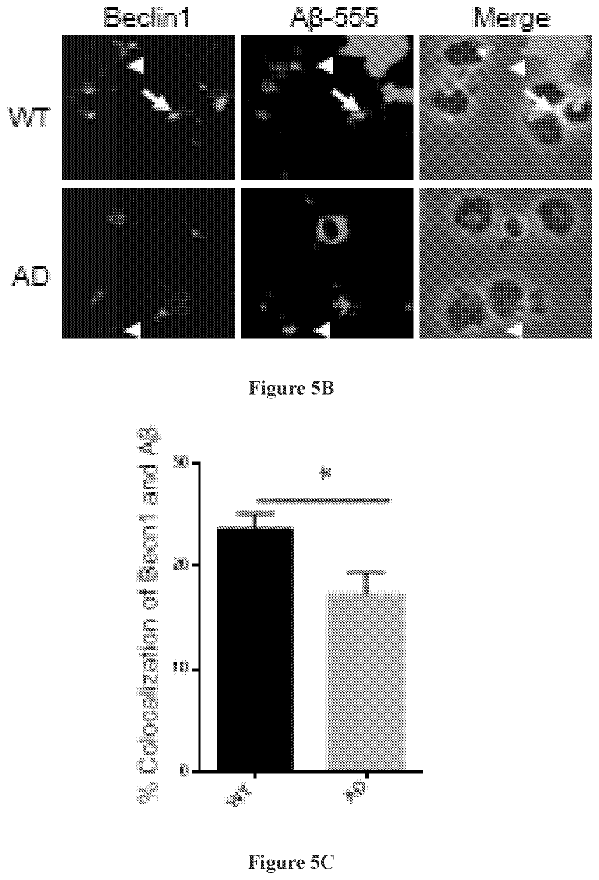

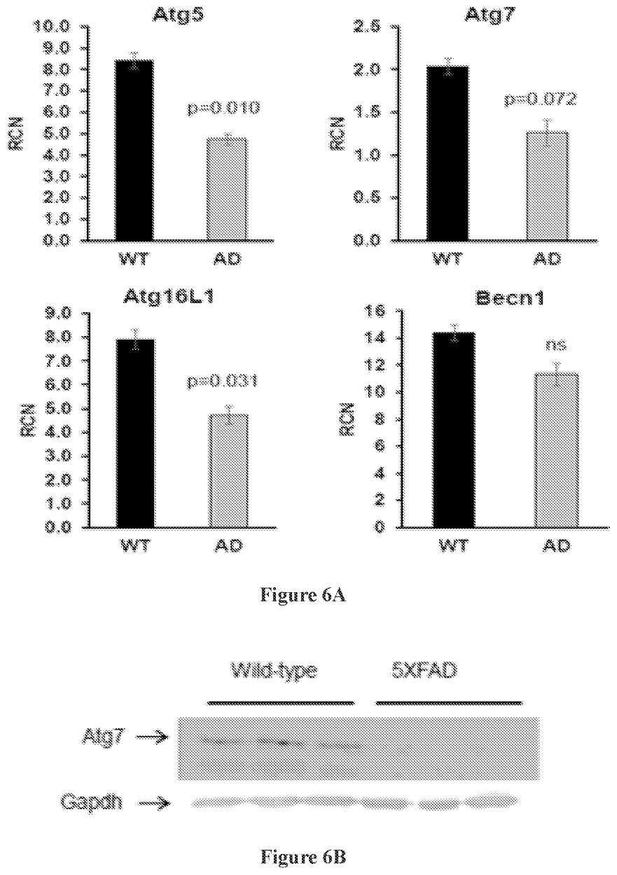

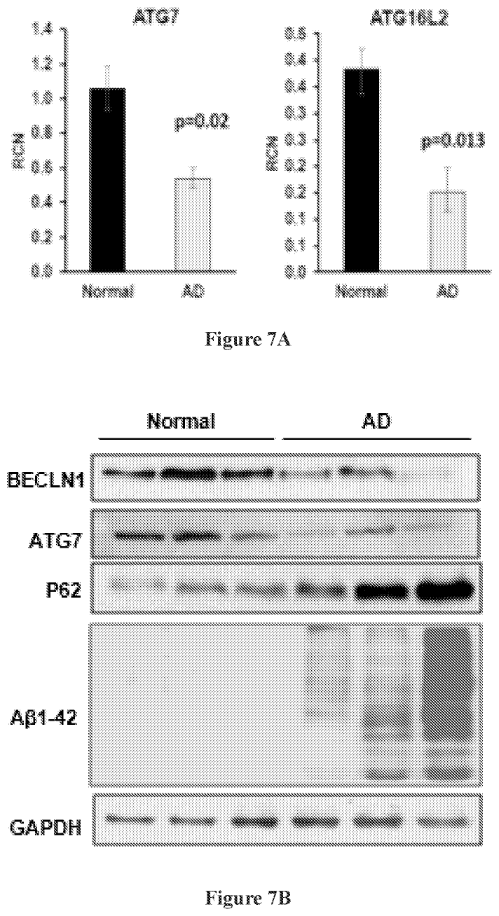

Microglia are the brain's resident immune cells that maintain tissue homeostasis by phagocytosis and degradation of dead cells, debris and unwanted molecules such as extracellular Aβ. The morphology of microglia differ according to their state of activation. They can be amoeboid or ramified. Ramified microglia, are homeostatic surveying microglia, characterized by thin processes extending out from a relatively circular-shaped soma (FIG. 4). Amoeboid microglia are more rounded, highly motile and can be found in inflammatory and phagocytic conditions (FIG. 4). Our data show that microglia isolated from 5×FAD mice display defective degradation of Aβ when compared to microglia isolated from wild-type (WT) healthy mice despite comparable uptake (FIGS. 2 A-C and FIG. 3). Notably, the degradation of Aβ is improved when AD microglia are treated with the autophagy stimulator rapamycin (FIG. 3), whereas WT microglia fail to degrade Aβ when autophagy is inhibited with 3-MA (FIG. 3). These data strongly support the role of autophagy in Aβ degradation by microglia. Further, vacuoles containing phagocytosed Aβ acquire the autophagy markers beclin1 and LC3 in primary microglia from WT mice, but not in AD microglia (FIG. 5 A-C). A defect in autophagy in AD is further supported by our novel finding that the expression of several autophagy mRNAs and corresponding proteins, Nbr1, beclin1, Atg5, Atg7 and Atg16L, are significantly down-regulated in mouse AD microglia (FIG. 6A-6B). Importantly, the same autophagy molecules are down-regulated (at the mRNA and protein levels) in the brain of human AD subjects when compared to age- and sex-matched individuals (FIG. 7A). Notably, the down-regulation of the autophagy effectors is detected in AD human brain sections at locations enriched with Aβ 38. Together, these findings demonstrate that Aβ are trafficked within autophagosomes in microglia and that autophagy plays a critical role in the degradation of Aβ in healthy microglia cells, while defective autophagy in AD microglia contributes to the lack of Aβ clearance.

The implication of dysfunctional autophagy in the pathogenesis of AD has been investigated. Initial studies have focused on neuronal cells. A noncanonical role for autophagy marker LC3 in the uptake of Aβ has been suggested, but the contribution of autophagy to Aβ degradation has not been investigated. AD microglia fail to endocytose Aβ and recycle surface receptors. However, there has been little investigated of the mechanism underlying the defective autophagy activity in microglia from adult AD mice. Using adult microglia, we have evaluated the dysfunctional microglia residing in the AD environment. Since isolation and culture may affect microglial properties and epigenetic signature, we demonstrate that the adult AD microglia cultured for several days still maintain elevated expression of the miR-17 and reduced expression of autophagy targets similar to those in fixed brain sections. Therefore, our method of isolation and culture of adult microglia offers a suitable model to study miR-17 in microglia in vitro.

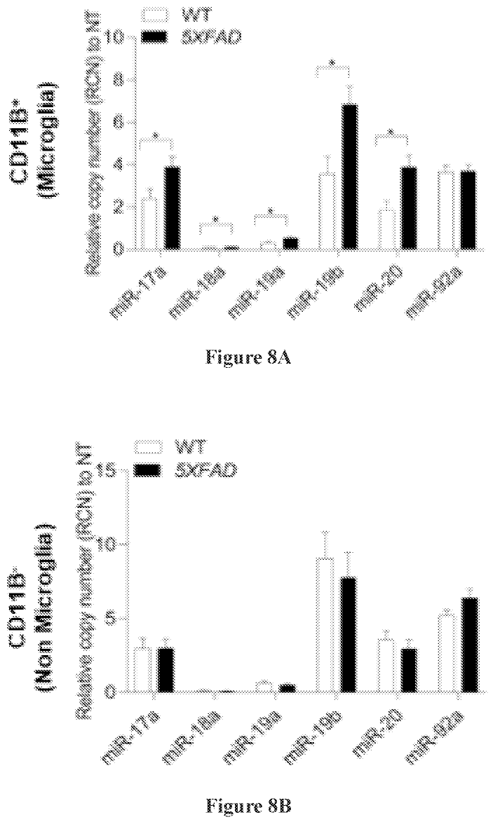

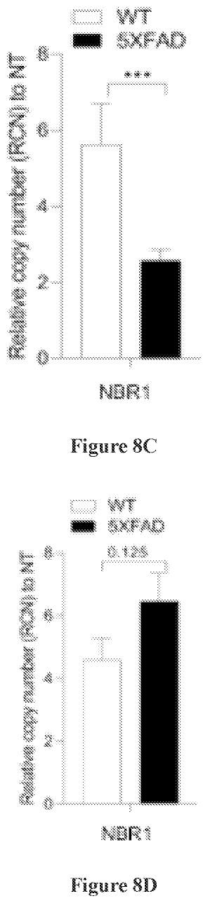

Numerous epigenetic changes have been detected in Alzheimer's disease (AD), and there is strong implication of microRNAs (miRs) both in autophagy and AD, yet few reports have linked autophagy and miRs in the context of AD. Inspired, we searched for miRs that regulate autophagy and which expression is altered in AD brains. miRs are evolutionarily conserved noncoding RNAs of 17-24 nucleotides, which mediate post-transcriptional gene silencing by binding to the 3′-untranslated region (UTR) or open reading frame (ORF) region of target mRNA. The expression and function of miRs are regulated at three levels: transcription, processing, and subcellular localization (FIG. 1). Specific miRs are strongly associated with various diseases and can be detected by as RT-qPCR. Using online web servers TargetScan and miRBase, we found that members of the miR-17-92 cluster repeatedly target essential autophagy molecules48. The miR-17-92 cluster consists of miR-17, miR-18a, miR-19a, miR-20a, miR-19b-1, and miR-92a-1 (FIG. 1, Panel B), and is conserved among vertebrates, and has been associated with roles in cell cycle, tumorigenesis, and aging. The expression of the members of the miR-17-92 cluster is downregulated during aging and senescence. Notably, in here we found that the expression of miR-17-92 cluster increases mainly in microglia in the brains of AD mice as they age but not in their WT counterparts and not in other cell types (FIG. 8A-8D). Therefore, our approach to analyze isolated microglia helped us detect differences that would have been masked if using whole brain homogenates. Similar to what we observed in the mouse microglia, the upregulation of miR-17-92 was very prominent in microglia of AD patients, when compared to their non-AD age- and sex-matched controls. In addition, the increased expression of miR-17-92 cluster correlated with reduced expression of its predicted targets ATG7, BECLIN1, and NBR1 in human and mouse brains (FIGS. 6A-6B, 7A-7B, and 8A-8D) and humans. Another very important finding is that miR-17 expression is elevated in human and mouse microglia adjacent to Aβ plaques, but not in microglia away from the plaques in the same section.

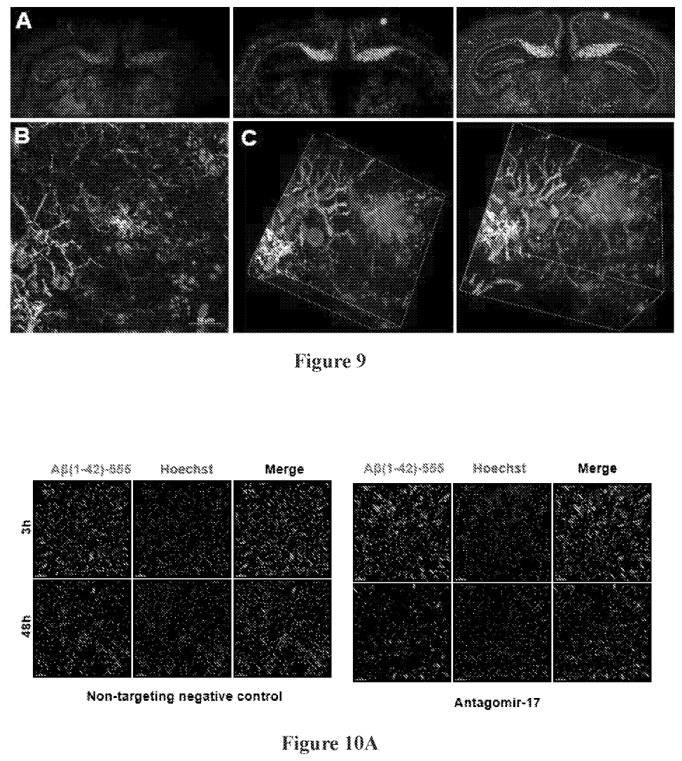

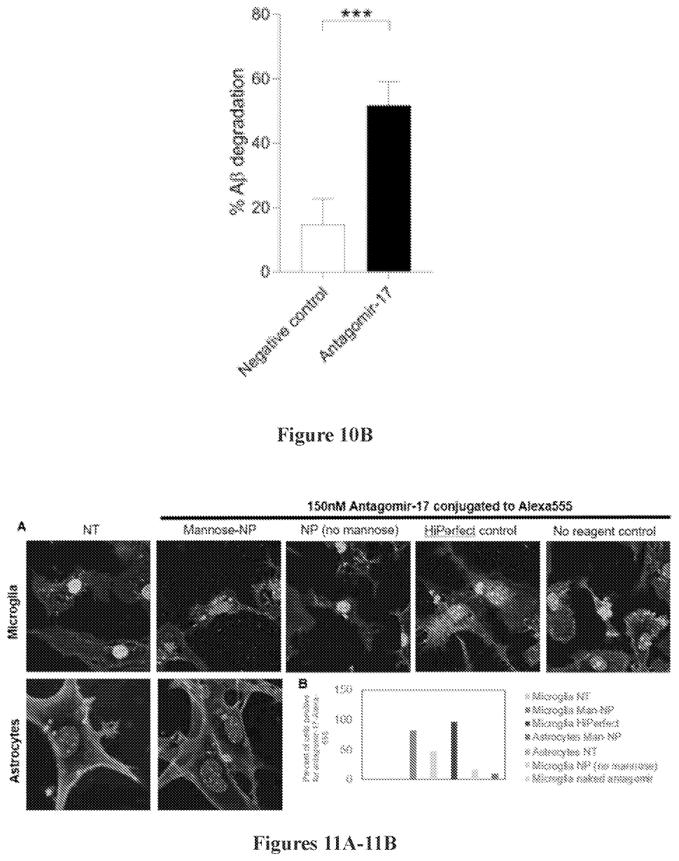

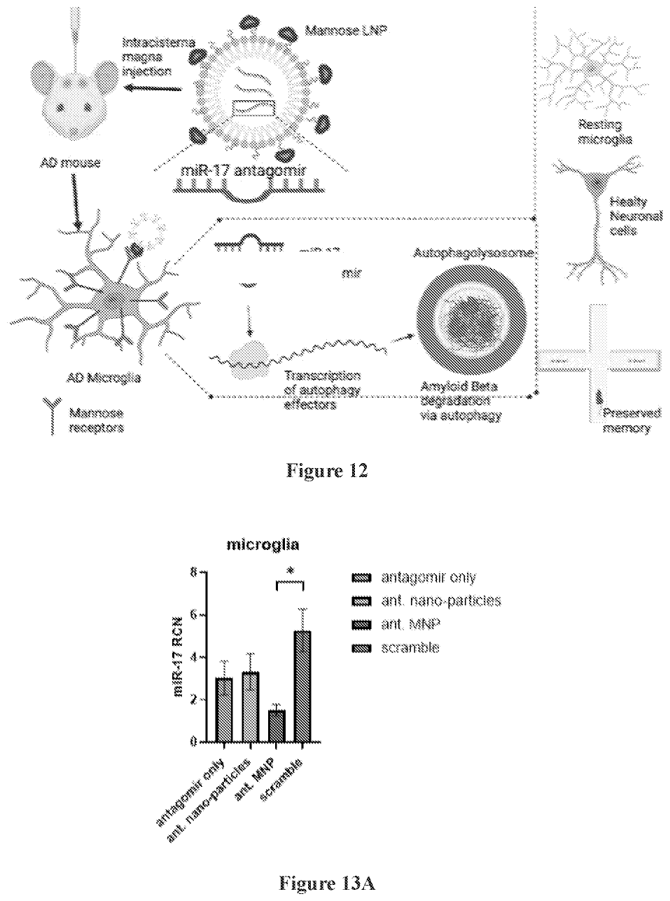

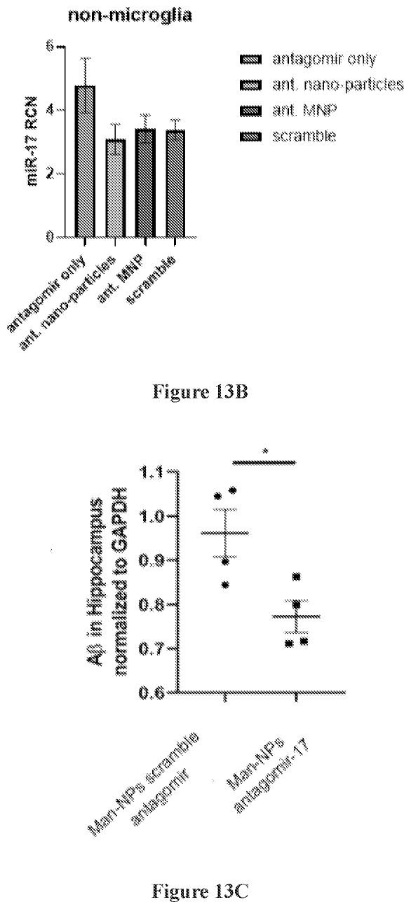

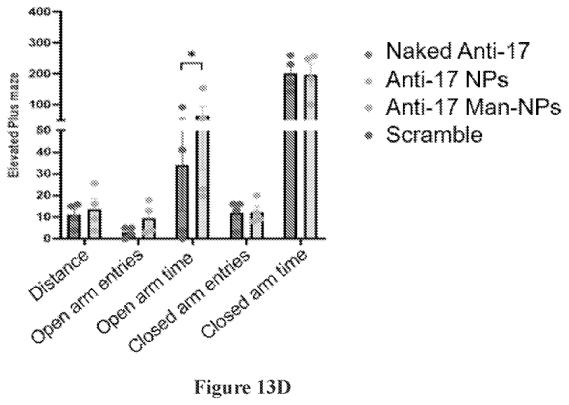

Together, our data led to the conclusion that elevated miR-17-92 cluster occurs in microglia residing in Aβ-rich areas of the brain due to prolonged exposure to Aβ. Notably, reducing the expression of miR-17 alone with specific antagomir in AD microglia improved Aβ degradation (FIG. 10A-10B). Interestingly, injection of miR-17 antagomir Man-LNPs for only 1 month, reduced Aβ levels in the brains of mice (FIGS. 12 and 13A-13C). Those injected with scramble antagomirs within Man-LNPs were not affected. Hence, we focused on miR-17 as a highly promising new therapeutic target. We also rely on our success in reducing the expression of miR-17 using Man-LNPs for the delivery of the miR-17 antagomir (FIG. 12) and reducing Aβ levels (13A-13C)

### Significance and Strategy

The FDA recently approved Adulhem (aducanumad), however, its efficacy will take years to confirm and will not be readily available to all patients. Its efficacy can also be improved with additional strategies. Thus, AD remains a major leading cause of death in the US, that cannot be prevented, cured, or delayed and is overwhelming for the patient and family members. AD brain pathology is characterized by build-up of Aβ plaques and neurofibrillary tangles of tau protein. Both adult WT and AD primary microglia similarly phagocytose Aβ but the AD microglia fail to degrade it (FIG. 2A-2C). In WT microglia, Aβ is contained within vacuoles that acquire specific autophagy markers (FIG. 5A-5C). Stimulation of autophagy in AD microglia with rapamycin improves Aβ degradation (FIG. 3). Therefore, our data support a major role for autophagy in the degradation of Aβ in primary adult microglia. The fact that the miR-17 cluster is elevated mainly in microglia in human and mice with AD is an important findings. Analysis of whole brain homogenates demonstrate equal low amounts of miR-17-92 in AD and non-AD brains because the high level in microglia alone was masked by low levels in other cells. Thus, this requires special microglia targeted therapies, which we propose herein. Together, the compositions, methods, and strategies described herein will improve our understanding of AD pathobiology, determine the mechanism of microglia dysfunction in AD, provide therapeutic targets for AD, and nanoparticles that can specifically deliver therapeutics to microglia in vivo. Many MicroRNA based therapeutics are being tested in phase II clinical trials which shows that they will soon be available for several disease conditions.

MiR-17 is upregulated in the microglia from the brains of AD patients. Notably, in silico analysis suggests that miR-17 targets autophagy effectors Nbr1, Atg5, Becn1, Atg7, Atg12 and Atg16L1 in human and mice. MiR-17 directly binds 3′UTR regions of Atg7 and Atg16L1 using luciferase assay; its binding to the rest of the targets can be similarly evaluated. Importantly, reducing the expression of miR-17 in the AD microglia improves Aβ degradation and Nbr1 expression. Our findings in the human AD brain are mirrored in the AD mouse model and hence makes it a great tool for in vitro and in vivo analyses. Remarkably, in vivo injection of miR-17 antagomir (Anti-17) Man-LNPs reduces Aβ levels in the brains of AD mice (FIG. 13A-13C). Autophagy markers can be colocalized with Aβ using a NIKON SIM-S super-resolution microscope offering 80 nm resolution (see FIGS. 9A-9C and 10A-10B).

Fluorescence microscopy and high-speed 3D fluorescence imaging using z-piezo stage and short time intervals (0.5-5 sec) can be used to image in real time the trafficking and degradation of fluorescent Aβ inside microglia. The Huygens and Metamorph software can be used to allow detection of fluorescent particles and characterize their shape, velocity and directionality, in addition to IMARIS analysis. These techniques will allow the precise detection and quantification of Aβ, and unbiased spatial determination of their colocalization with markers. In order to visualize and quantify mRNA of autophagy effectors in brain sections, we will use RNAscope in situ hybridization, state-of-the-art 2-D and 3-D quantitative fluorescence imaging. We will also use immunohistochemistry with the new RNAscope (Acdbio) to detect and quantify miR-17 within brain sections. Notably, we will use Hiperfect transfection reagent for primary cells (Qiagen) in vitro, which significantly facilitates the transfection of primary microglia without affecting their activation or morphology. With this approach, we can demonstrate that the addition of miR-17 antagomir to primary AD microglia restored their ability to degrade Aβ. For in vivo antagomir administration, we will use the minimally invasive intra-cisterna magna (ICM) injection technique which requires no surgery, gets directly into the CNS, and can be performed repeatedly (FIG. 13A-13C). Mannose-labeled nanoparticles (Man-LNPs) can be used to specifically target the mannose receptor on microglia for the specific delivery of an miR-17antagomir to microglia (FIG. 13A-13C). This can avoid off-target effects of miR-17 antagomir (Anti-17). The Man-LNPs can comprise of Man-PEG-DSPE/PEGDMG/DLin-MC3-DMA/DSPC/Chol. The Man-LNPs can be synthesized by microfluidic self-assembly and characterized for particle size, zeta potential, antagomir encapsulation percentage, and efficiency and selectivity of target modulation. The mannose receptor is mainly expressed on immune cells including microglia while not detected on neurons. Mannose receptor is also present in a functional state in rat microglial cells. Alexa Fluor-tagged Anti-17 can be used as a probe to facilitate study via fluorescence imaging.

Our data incorporated demonstrate the effective delivery of therapeutic molecules (Anti-17) specifically to microglia in vitro and in vivo (FIGS. 11A-11B, 12, and 13A-13C). Together, our data support the conclusion that elevated miR-17 in AD microglia provokes down-regulation of autophagy effectors, which prevents microglia from efficiently clearing A05. The delivery of anti-17 specifically to AD microglia using Man-LNPs reduced Aβ accumulation in the AD brain.

Investigation of the Mechanisms that Upregulate miR-17 and Affects Autophagy Targets in AD Microglia

Determination of the effect of Aβ and/or Tau exposure on the expression of miR-17. MiR-17 expression is increased in microglia adjacent to Aβ plaques in human AD brain sections (FIG. 8A-8D). In the brains of AD mouse model, the upregulation of miR-17 was observed starting at 4 months of age by RT-PCR within microglia present in the Aβ-rich areas. Therefore, the common factor between the human and mouse brains is that microglia are chronically exposed to Aβ plaques. Thus, it appears that prolonged exposure to Aβ leads to the upregulation of miR-17-92 cluster in microglia through specific receptor/s-mediated activation of signally pathways. Several receptors exist on the surface of microglia that can sense Aβ, mediate phagocytosis and/or mediate downstream signaling. Microglial cells adjacent to Aβ plaques express the scavenger receptors SCARA1 and SCARA2, which have a high affinity for soluble and fibrillar Aβ and mediate phagocytosis and clearance of Aβ from the brain. In addition, the macrophage receptor with collagenous structure (MARCO) binds Aβ and activates the ERK1/2 signaling pathway. TLR2 interacts with Aβ, RAGE115, CD36 and FPR2, activates microglia following Aβ binding. This is further supported by the finding that prolonged exposure of microglia for several hours to Aβ reduced autophagy activity.

To evaluate the effect of Aβ and/or Tau exposure on the expression of miR-17, WT microglia will be treated for 48-96 hrs with commercially available monomeric and fibrillary Aβ (Anaspec) at 1 μM. Controls will be treated with scramble Aβ. Then, the expression of the miR-17-92 will be determined by qRT-PCR. Commercially available Aβ can be used; alternatively, Aβ plaques can be purified from the brains of the AD mice or frozen human brains. Since Tau tangles are often present in the areas where Aβ accumulates, phosphorylated Tau (StressMarq Biociences) can be added to WT microglia to determine if Tau will increase the expression of miR-17-92. We will use available KOs or down-regulate the expression of individual receptors that are not available in primary microglia using shRNA and CRISPR/Cas9 gene knockdowns (KD) and knockouts (KO), using lentiviral delivery system122. Scramble shRNA will be used as control. We will treat the KO microglia asnin described above and identify the receptor(s) that mediate increased expression of miR-17-92.

Identifying the factors that control transcription of miR-17-92 cluster. The miR-17-92 cluster is a polycistronic cluster that has a single open reading frame located over ˜800-nt within chromosome 13 in humans. RNA pol II generates capped pri-miRNAs which are processed by Drosha and DGCR8 in the nucleus to generate pre-miRNAs. After translocation into the cytoplasm by exportin-5, pre-miRNAs are processed by Dicer to form the mature miRNA/miRNA (FIG. 1). The mature miRNA directs repression of mRNA containing partially complementary miRNA binding sites within the 3′UTR. The expression of miR-17-92 cluster is regulated by the transcription factors c-Myc and E2F1 (FIG. 1, Panel B). The activity of c-Myc is regulated by its expression level and phosphorylation status at serine 62 (S62) and threonine 58 (T58). Phosphorylation of S62 by ERK kinases transiently increases c-Myc stability whereas phosphorylation of T58 by GSK30 triggers proteosomal degradation. On the other hand, E2F1 is a validated target of miR-17 and miR-20 which in turn regulates the expression of miR-17-92 cluster. To determine the contribution of ERK, c-Myc and/or E2F1 activation or upregulation to increased miR-17-92 expression in AD, we will perform the experiments below.

To uncover the factors controlling the transcription of the miR-17-92 cluster in AD microglia, we will measure the expression of RNA pol II, Drosha, DGCR8, exportin-5 and Dicer (FIG. 1, Panel A) by western blots. We will examine the expression of the transcription factors c-Myc, and E2F1. We will also examine c-Myc activation by measuring its phosphorylation. These analyses will be performed in the brains and isolated microglia from AD mice (5 months) and human patients with AD (65 years and above). To determine the effect of post-transcriptional phosphorylation of c-Myc on the expression of miR-17-92 cluster, we will use AD mice adult microglia and apply specific inhibitors of the MEK/Erk1/2 (1-10 μM U0126 and the newly FDA approved MEK inhibitor Trametinib). Control experiments will include the inactive analogue U0124 (1-10 μM). To examine the effect of ERK inhibition, treated cells will be lysed and subjected to western blot analysis with antibodies against phospho-Erk1/2-(Thr202/Tyr204) and expression of the cluster will be determined by RT-PCR. To determine the effect of Erk kinase inhibitors on the expression and phosphorylation (activation) of c-Myc, treated cells will be analyzed by western blots using c-Myc and phospho-c-Myc antibodies. Then, to determine the effect of c-Myc phosphorylation, we will examine the effect of c-Myc inhibitor 10058-F4 that prevents c-Myc-Max dimerization and transactivation of c-Myc target gene expression on microglia isolated from AD mice. We will also use HLM006474 which is a pan E2F inhibitor to establish if E2F controls miR-17 expression. In addition, we will determine phospho-ERK, phospho-c-myc, and E2F. These experiments will be performed in brain tissues from AD and non-AD mice and human subjects.

Identifying the 3′UTR region of autophagy effectors bound by miR-17 and the direct consequence of this binding on autophagy transcript and protein expression. miRs post-transcriptionally regulate gene expression, predominantly through imperfect base pairing with the 3′-untranslated region (3′UTR) of target mRNA. To confirm that miR-17 binds to its predicted targets, we performed a luciferase assay which showed that miR-17 directly binds the 3′ UTR of Atg7 and Atg16L. The absence of the miR-17-92 cluster is associated with increased expression of Atg5 in macrophages, whereas the increased expression of the cluster is associated with reduced expression of NBR15, Atg7 and Atg5. These data confirm in silico predictions and provide evidence that miR-17 modulates the expression of Atg5, Atg7 and Atg16L. To further demonstrate that miR-17 specifically binds to additional targets Nbr1, beclin1 and Atg12, we will analyze its binding to their target 3′UTR by Luciferase.

Results. We expect that prolonged exposure of WT microglia to commercial or Aβ isolated from AD mice will increase the expression of the miR-17-92 cluster in microglia. Using KO/KD of different Aβ receptors will allow us to identify the receptor/s responsible for mediating the signal. If the suggested individual receptors known to bind Aβ contribute to partial upregulation of the cluster, we will generate double KOs. If none of them contributes to the upregulation of miR-17-92, we will examine the role of other major receptors such as TREM2. TREM2 protein is expressed on microglia, binds to soluble oligomers of Aβ and promotes phagocytosis, modulates inflammatory signaling, and promotes microglial survival. Although TREM2 polymorphisms are associated with a risk for late onset AD, its role in neurodegenerative diseases is controversial. In the unlikely event that prolonged Aβ exposure does not affect the miR-17-92 cluster, we will determine if the culture supernatants of neuronal cells or astrocytes treated with Aβ will instigate the upregulation of the miR-17-92 cluster in microglia. If this is the case, we will do a proteomic analysis of culture supernatants and identify the factors provoking the upregulation of the miR-17-92 cluster in microglia. We also expect that in AD microglia, c-Myc will be mainly phosphorylated at S62, which will suggest that it is phosphorylated by ERK and this contributes to its stability. We anticipate that inhibition of ERK will decrease phosphorylation of c-Myc resulting in subsequent reduction of the expression of miR-17-92 cluster. Accordingly, we expect an increase in the expression of autophagy molecules. This result will prompt us to examine the effect of ERK inhibitors in vitro and in vivo in comparison with antagomirs and establish the best approach for restoring autophagy function in microglia, improving Aβ degradation and preventing AD pathobiology with least off-target effects. We expect to find that E2F1 is upregulated in AD microglia, which leads to increased expression of the miR-17-92 cluster. If this is not the case, we will determine the expression of E2F2 and 3 since few emerging reports, determined that they contribute to the expression of the cluster. We expect that the expression of Drosha, DGCR8 and Dicer is altered in AD samples. We will also examine if other factors such as CDSF3 and ISY1 contribute to the biogenesis of the miR-17-92 cluster and their expression is altered in AD as they do in other conditions including cancer. In addition, MiR expression can be controlled by DNA methylation, and activity of transcription factors. However, we have performed methylation analysis of AD brain samples and the miR-17-92 cluster was not differentially methylated. We also expect to observe that miR-17 interacts with the 3′UTR of Nbr1, beclin1 and Atg16L.

Determining the Functional Consequences of Reducing the Expression of miR-17 In AD Mice In Vivo

Determining the effect of Anti-17 Man-LNPs injection on the expression of miR-17 autophagy targets. We have found that miR-17 is upregulated in microglia within the brains of AD humans and mice when compared to age- and sex-matched controls5 (FIGS. 8A-8D). The upregulation of the miR-17 in AD microglia is accompanied by reduced expression of the predicted autophagy targets Nbr1, beclin1, Atg5, Atg7 and Atg16L5. In vitro data demonstrated that treatment of primary AD microglia with naked Anti-17 without Man-LNPs significantly reduces the expression of miR-17 and improves the degradation of Aβ, with increased expression of Nbr1 and formation of autophagosomes. When naked Anti-17 was injected in vivo, miR-17 was reduced in all cell types (FIGS. 10A-10B), however, Aβ accumulation was increased in the brains of injected mice when compared to scrambled control. This was observed because miR-17 expression was reduced in vivo in all cell types including neuronal cells where miR-17 also targets Aβ precursor protein (APP). This led to increased APP expression (since APP is one of the predicted targets of miR-17) in neuronal cells, which increased Aβ deposition. Hence, it can be important to deliver the Anti-17 only to microglia to prevent the off-target effects that occurs in vivo.

To selectively target microglia, the Anti-17 was loaded into mannose-conjugated lipid nanoparticles (Man-LNPs) that serve as delivery vehicles. The mannose receptor is mainly expressed on immune cells including microglia while not detected on neurons. Mannose receptor is also present in a functional state in rat microglial cells. The Man-LNPs, comprising Man-PEG-DSPE/PEGDMG/DLin-MC3-DMA/DSPC/Chol, can be synthesized by microfluidic self-assembly and characterized for particle size, zeta potential, antagomir encapsulation percentage, and efficiency and selectivity of target modulation.

Man-LNPs were added to primary microglia and astrocytes with and without their cargo (Alexa Fluor-tagged Anti-17). A transfection reagent was used as a positive control and no reagent or NPs served a negative control. FIGS. 11A-11B and 13A-13C show that Man-LNPs preferentially deliver their cargo (Alexa Fluor-tagged antagomir-17) to microglia in vitro and in vivo.

We injected 4 groups of AD mice (each made up of 4 mice, 2 males and 2 females). Mice were injected with either Anti-17 alone, Anti-17 in LNPs without mannose, Anti-17 within Man-LNPs, and scramble antagomir within Man-LNPs (FIG. 13A-13C). AD mice were injected once weekly for 4 weeks starting at the age of 3.5 months (4 μl total volume injected at concentration of 0.3 nmole/μl) then microglia and non-microglia fractions were isolated (FIG. 13A-FIG. 13B). They were analyzed by RT-PCR to determine the level of miR-17. Brain homogenates were analyzed for Aβ by western blots. Densitometry of Aβ band was normalized to GAPDH (FIG. 13C). Notably, Anti-17 packaged within Man-LNPs were able to significantly reduce the expression of miR-17 and this was achieved only in the microglia fraction. In addition, Aβ was reduced significantly in the brains of the Anti-17 Man-LNPs treated mice (FIG. 12). These results support the conclusion that in vivo delivery of Anti-17 within Man-LNPs will reduce the expression of miR-17 mainly in microglia hence will have minimal off-target effects and will improve the expression of its autophagy targets in microglia without affecting the expression of APP in neuronal cells.

As described above, mice will be briefly anesthetized with isoflurane; the dorsal aspect of the skull will be shaved and swabbed with 70% EtOH. A 27-gauge needle connected to a Hamilton syringe via PE50 tubing will be inserted into the cisterna magna. To verify entry into the cisterna magna, 2 μl of clear CSF will be drawn and gently pushed back in prior to slow injection of 4 μl of Anti-17 within Man-LNPs and allowing 1 min before the needle is removed. The injections will be administrated once a week for 4 weeks. We will start the injections to 3.5-month old AD mice. One group will be injected with Anti-17 Man-LNPs, a second group will receive scramble control antagomir within Man-LNPs, a third group will receive empty Man-LNPs and a forth group will be mockinjected. Then, mice will be sacrificed. We will measure the level of miR-17 and its autophagy targets in microglia CD11b+ and non-microglia CD11 b-fractions by RT-PCR and by immuno-histo-chemistry. Using this strategy, we will determine the expression and distribution of Atg5, Atg7, beclin1 and Atg16L in situ in brain sections with focus on microglia using combined in situ hybridization and immuno-histochemistry with RNAscope and specific antibodies. The brains of mice treated with scramble antagomir, empty LNPs and mockinjected mice will be used as controls.

Determining if injection of Anti-17 Man-LNPs in vivo reduce microglia activation and neuronal loss. The injection of Anti-17 Man-NPs will improve AD pathology including reducing the accumulation of Aβ, the activation of microglia and the loss of neurons in the brains of AD mice. To assess these results, we will examine the expression of Aβ and its distribution within the brain by western blot and confocal microscopy after in vivo Anti-17 Man-LNPs treatment to test if reduction of miR-17 is accompanied by reduction of Aβ deposition. We will analyze the activation of microglia using image capture and analysis. Proliferation and morphology will be determined using Iba1 and morphologic analysis, skeletonization, processes analysis. Arborization analysis will be achieved by “Filament” function in IMARIS software. Microglia activation will be determined by measuring mRNA levels of CD11b, iba1, MHCII, CD86 by RT-PCR153. Neuronal morphology will be examined using Cresyl violet staining while degenerated neurons will be identified using Fluor-Jade C staining. Quantification of dendrites will be performed using Golgi-Cox staining. We will quantify synaptic markers including PSD95, Shank, Homer, Drebrin, Kalirin-7, CREB, and phosphorylated CREB (pCREB), presynaptic vesicles using Synaptophysin, neuronal cell levels using NeuN 153. Images will be acquired using Olympus FV3000 microscope and NIKON SIM-S super-resolution microscope 100× magnification. 3D reconstruction and image analysis will be performed by IMARIS software using the “surface rendering” and “spot detection” functions.

Determining if injection of Anti-17 Man-LNPs in vivo prevents or delays the deterioration in memory of AD mice. We will confirm that injection of Anti-17 Man-LNPs reduces or delays the deterioration of memory of AD mice. For behavioral studies in AD mice, we will employ the Y maze and Barnes maze tests to examine spatial memory performance because AD patients are reported to have poor spatial memory and navigation. The Y maze examines spatial working memory and requires a single 5 min testing trial. The mice have free access to the maze and make choices on their own. The Barnes maze examines long-term spatial reference memory and requires up to 8 days of repeated testing. Each testing day includes 3 test trials. Here the mice use constant spatial cues around the maze to locate a goal box. Compared to the water maze, Barnes maze is the preferred tool for assessing spatial learning and memory changes in AD mouse models and is less stressful.

Results. We expect that our approach will be successful in reducing the expression of miR-17 in microglia of AD mice. Although unlikely, in case the delivery of antagomirs by ICM injections is not successful, or causes problems due to repeated injections, we will perform it stereotaxically. As described above, we will determine the effect of Anti-17 Man LNPs within 1 week of the last ICM injection. In addition, we will also determine the duration of Anti-17 Man-LNPs effect in mice by examining the expression of miR-17 in microglia 1, 2 and 4 months after the last ICM injection. We will also reduce the frequency of injections to once every 4 weeks if the effect of the antagomir is stable for 4 weeks. The delivery of antagomirs through injections, whether via ICM or stereotaxis is for proof of principle, and will open the possibilities for the future design of other routes of delivery, that are less invasive. We also expect that the expression of Atg5, Atg7 and Atg16L will markedly increase upon the down-regulation of miR-17. The most important desirable outcome is the reduction of Aβ accumulation and memory loss even if the expression of autophagy effectors does not improve significantly. Although unlikely, in this case we would examine the other miR-17 targets to identify which ones are responsible for the improvement of AD pathobiology.

Determining the Phenotype of Microglia Isolated from AD Mice after In Vivo Injections of Anti-17 Man-LNPs

Determining if the Aβ-containing vacuoles acquire autophagy markers and mature into autophagolysosomes. We will also assess if injection of Anti-17 Man-LNPs in AD mice will enhance the localization of Aβ within autophagosomes and improve autophagy flux ex vivo, in isolated microglia.

Injections will be performed as explained in FIG. 12 and performed in FIGS. 10A-10B and 13A-13C. We will compare the same groups described above. Microglia will be isolated from all mice groups within 1 week of the last injection. We will determine if the Aβ-containing vacuoles acquire autophagy and lysosomal markers. Cells will be incubated with fluorescent FL-Aβ (Aβ-555) for 30 min, washed and incubated for up to 12 h, followed by chemical fixation. Cells will be permeabilized, and labeled with fluorescent antibodies directed against markers for early (EEA1, Rab5) and late (Rab7, LAMP1/2) endosomes, and for autophagosomes (LC3, Beclin 1, Atg7, Atg5). This will allow monitoring and quantifying the maturation of Aβ-containing vacuoles by measuring their colocalization with the specific markers by fluorescence microscopy. Autophagy activity (flux) which will be determined by western blot for LC3 conversion in the presence of Bafilomycin A.

Determining if isolated microglia regained the ability degrade fibrillar As in functional lysosomes. Treating AD microglia in vitro with Anti-17 significantly improves their ability to degrade Aβ (FIGS. 10A-10B). In addition, specific delivery of Anti-17 Man-LNPs to microglia reduces Aβ levels in the brains of AD mice (FIG. 13A-13C). These results indicate that targeting miR-17 alone is sufficient to restore the ability of AD microglia to efficiently clear Aβ. We will confirm that the delivery of Anti-17 Man-NPs in vivo will restore the ability of isolated adult AD microglia to degrade Aβ ex vivo.

We will isolate microglia from the mice groups described above and: (I) Determine the M1 versus M2 phenotype of microglia by measuring expression specific markers (CD206, CD204, Arg1, CD68, CD16, CD86, iNOs) by western blotting and flow cytometry, and measure the production of cytokines from the cell culture supernatant (IL-10, IL-13, IL-4, TNFα, IL-6, IL1β)171. (II) Determine if in vivo injections of Anti-17 Man-LNPs in AD mice improves Aβ degradation. Aβ degradation will be measured by Aβ(1-42) ELISA and by fluorescence microscopy (FIGS. 2A-2D and 10A-10B). Microglia will also be evaluated for phagocytosis and degradation of fluorescent-Aβ-HiLyte-488 by fixing microglia after 1 and 48 hrs of incubation, followed by staining for extracellular Aβ (CSTD54D2), and quantification of changes in intracellular and extracellular Aβ fluorescence intensity and by ELISA. (III) Determine if Aβ colocalizes with lysosomes in microglia. Lysosomes will be visualized by preloading cells with fluorescent lysotracker or fluorescent dextrans, which traffic and accumulate in acidic lysosomal compartments. Confocal fluorescence microscopy analysis and live cell imaging will be performed with appropriate spatial resolution to discern the co-localization between Aβ-555 and lysosomes. Additionally, we will use non-fluorescent Aβ that can be labeled post-fixation. We will use the Huygens software to measure the co-localization index in 3-D after deconvolution of the confocal images. (IV) Measure lysosomal pH in microglia. Several studies tested the acidification of lysosomes in neurons, blastocysts and fibroblasts from different AD mouse models, but no studies examined lysosomes in primary microglia from the 5×FAD mice. The acidification of lysosomes will be determined in the presence and absence of non-fluorescent Aβ. Microglia will be incubated with LysoSensor™ Yellow/Blue DND-160, a ratiometric pH indicator that localizes to acidic compartments. Microglia will be imaged using SpectraMaxi3× micro-plate reader that has dual spectroscopy and fluorescence imaging capabilities (Molecular Devices). Data will be expressed as a fluorescence ratio, which indicates the pH, and the number of the cells will be counted by automated phase-contrast analysis. As a second method to measure the lysosomal pH, we will perform pulse/chase experiments with Dextrans conjugated to Fluorescein and Tetramethylrhodamine (TMR, 70 kDa). The ratio of fluorescence from pH-sensitive Fluorescein and pH-insensitive TMR allows for the measurement of lysosomal pH. (V) To measure the pH of Aβ-containing vacuoles, we will conjugate Aβ with the pHrodo iFL Red then allow phagocytosis and follow acidification of the Aβ-containing vacuole. iFL Red dye fluorescence dramatically increases at acidic pH. (VI) Measure lysosomal proteolytic activity in microglia. Microglia will be loaded with DQ Green-BSA, chased for 3 h to allow accumulation of BSA in lysosomes. Enzymatic degradation of the BSA leads to fluorochrome dequenching and fluorescence emission. Normalization will be performed based on the relative level of DQ Green-BSA uptake by microglia. We will then repeat the experiments using quantitative live-cell imaging at selected time-ranges to refine our analyses and determine the spatiotemporal dynamics of vacuole maturation, their cytosolic trajectories and trafficking in real time.

Characterizing the phenotypes of isolated microglia using single cell RNAseq analysis. The increased expression of miR-17 and accompanying low expression of autophagy targets is more pronounced in microglia adjacent to the Aβ plaques in human and mice. Therefore, these findings suggest that several subsets of microglia exist within the AD brain. Accordingly, we will characterize the properties of different microglia with the brain of AD mice before and after Anti-17 Man-LNP injections.

We will dissect the different regions of the brain of mice injected with Anti-17 Man-NPs, scramble-Man-LNPs, non-injected AD and WT mice. The regions include prefrontal cortex, thalamus, hippocampus, and brain stem. Cell type specific gene expression will be compared across different brain regions that will be performed by GENEWIZ. We will perform Spatial Mapping of Gene Expression at Single Cell Resolution with the RNAscope® Technology Confirm scRNA-seq results within the tissue context.

Results. We expect that after injection with Anti-17 Man-LNPs, Aβ will traffic within autophagosomes and then autophago-lysosomes in isolated AD microglia and will be degraded more efficiently, similar to WT microglia. We expect therefore, that Aβ-containing vacuoles will sequentially acquire the autophagy and lysosomal markers. In contrast, we expect that in AD microglia isolated from scramble control or mock-injected mice, Aβ-containing vacuoles will be defective in acquisition of autophagy markers and degradation of Aβ. Although the ideal time points for measuring the degradation of Aβ have been established, we may need to examine longer durations of injections to see significant effects of the antagomirs. We will also establish if these defects are associated with abnormal lysosomal pH or not, and if the autophagy defect is specific to Aβ, or also generally observed for other cargos known to be degraded by autophagy. It is possible that we will need to add an antagomir against another member of the cluster to improve the degradation of Aβ by AD microglia. It is probable that in vivo injection of Anti-17 will improve the Aβ hallmarks of AD mice, yet isolated microglia from these mice may not show drastic improvement of their functions. This may be due to minor improvement that is collectively more effective in vivo. It is also possible that the treatment with Anti-17 Man-NPs affected other signaling pathways downstream of miR-17, which we will identify and examine. Therefore, our work will confirm the role of miR-17 as a drug target for AD and Man-LNPs as viable candidates for delivery of therapeutic cargo to microglia.

Experimental Design and Methods. In our experiments we will use 5×FAD Mouse model (AD) which is a double transgenic APP/PS1 mouse model that co-expresses five AD mutations leading to accelerated plaque formation and it recapitulates many AD-related phenotypes observed in humans. Equal numbers of male and female mice will be used in our studies. Microglia from adult mice will be isolated as we previously described 7 by MACS® neural dissociation kit (Miltenyi Biotec) followed by CD11b magnetic bead (Miltenyi Biotec) isolation technique to positively select for brain microglia expressing the pan-microglial marker CD11b7. Mannose-labeled lipid nanoparticles (Man-LNPs) are formed from a lipid mixture comprising one or more lipids comprising a microglial targeting agent (e.g., Man-PEG-DSPE); one or more ionizable lipids, one or more cationic lipids, or a combination thereof (e.g., DLin-MC3-DMA); one or more neutral lipids (DSPC, cholesterol, or a combination thereof); and one or more PEGylated lipids (PEG-DMG). They can be synthesized by microfluidic self-assembly and characterized for particle size, zeta potential, antagomir encapsulation percentage, and efficiency and selectivity of target modulation.

The active agent can be a chemically modified antimir oligonucleotide that specifically targets miR-17, which is composed of fully complementary antimiR and a seed directed locked nucleic acid. The efficacy for inhibiting miR-17 was tested by its transfection into macrophages and miR-17 RCN was significantly reduced. miR-17 antagomir (1.2 nmole) will be delivered by Intra-Cisterna-Magna (ICM) injection as previously described 7,4. 3.5-month-old AD mice will be injected once every week for 4 months. Another group of mice will receive scramble control antagomir and a third group will be mock injected. Then, mice will be sacrificed within 1 week of the last injection.

Mannose-Amine-PEG-DSPE was prepared as outlined in the following steps:
1. The following reagents were prepared:
DSPE-PEG-NH2-3400 (Laysan Bio, Inc), 100 mg/mL in DMSO
α-D-Mannopyranosylphenyl isothiocyanate (Sigma, M9271), 10 mg/mL in DMSO.
2. 100 μL of DSPE-PEG-NH2-3400 was mixed with 30 μL of Mann-isothiocyanate at a molar ratio of 1:2, an additional 6.5 uL of triethylamine (TEA) (Sigma, 90335) was added as the catalyst, and the mixture was incubated at 37° C. for 1 hour.
3. The working solution of Mann-PEG-DSPE had an effective PEG-DSPE concentration of 73.26 mg/mL.

Man-LNPs were prepared as outlined in the following steps:
1. The following reagents were prepared:
   DODMA(BP-25707) (150 mg/mL in pure ethanol).
   DOPC (Avanti, 850375) (150 mg/mL in pure ethanol).
   20 mM Phosphate Buffer (PB) at pH 4.0.
   2N HCl.
   0.5N NaOH.
   Active agent Oligos (antimiR-17 and mismatched control) at 1 mM in DEPC water.

“MIXMER”: antimir-17 mixmer (all in PS bond):
(SEQ. ID No. 3)
5′-+C*+A*C*+T*G*+T*A*A*+G*+C*dA*C*+T*T*+T-3′
“MISMATCHED”: non-targeting control:
(SEQ. ID No. 4)
5′-+C*+T*C*+A*G*+T*A*A*+A*+G*A*T+A*T*+T-3′

2. 310 μL of 20 mM PB with 9 μL of 2N HCl was preheated to 65° C.
3. 44 μL of DOPC, 37 μL of DODMA, and 30.7 μL of Mann-PEG-DSPE were combined to form a lipid mixture.
4. On a vortex, rapidly inject the lipid mixture into the buffer using a 29G insulin syringe. Cap the vial and mix the solution well at a high-speed vortex. The product solution (empty LNP) should be clear and transparent.
5. Aliquot 50 μL of empty LNP in an Eppendorf tube. Dilute active agent (oligo) to 0.6 mM in DEPC water.
6. On a vortex, add 50 μL of oligo solution to the 50 μL empty LNP drop by drop. Mix well by vortexing for 10 seconds.
7. On a vortex, add 5 μL of 0.5N NaOH to the 100 μL LNP solution. The solution should turn semi-transparent. The Man-LNP solution and empty LNP solution can then be stored for up to 4 weeks at 4° C.

The compositions, and methods of the appended claims are not limited in scope by the specific compositions and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A pharmaceutical composition comprising a lipid particle encapsulating an active agent, the lipid particle comprising:
one or more lipids comprising a microglial targeting agent;
one or more ionizable lipids, one or more cationic lipids, or a combination thereof;
one or more neutral lipids; and
optionally one or more PEGylated lipids;
wherein the active agent inhibits the transcription or translation of a human miR17-92 cluster.

2. The composition of claim 1, wherein the active agent comprises a nucleic acid.

3. The composition of any of claims 1-2, wherein the active agent comprises an antagomir.

4. The composition of claim 3, wherein the antagomir comprises a nucleic acid that hybridizes to a human miR17-92 cluster under moderate stringent conditions.

5. The composition of claim 4, wherein the nucleic acid hybridizes to the miR17-92 cluster under high stringent conditions.

6. The composition of any of claims 4-5, wherein the nucleic acid hybridizes to miR-17, miR-18a, miR-19a, miR-20a, miR-19b, or miR-92.

7. The composition of any of claims 4-6 wherein the nucleic acid hybridizes to miR-17.

8. The composition of any of claims 4-7, wherein the nucleic acid hybridizes to SEQ ID Nos.: 1, 9, 13, 17, 21, or 25.

9. The composition of any of claims 4-8, wherein the nucleic acid hybridizes to SEQ ID Nos.: 1.

10. The composition of any of claims 4-9, wherein the nucleic acid has at least 80%, at least 90%, at least 95%, or at least 99% sequence homology to SEQ ID Nos: 3, 5, 7, 11, 15, 19, 23, or 27.

11. The composition of any of claims 4-10, wherein the nucleic acid has at least 80%, at least 90%, at least 95%, or at least 99% sequence homology to SEQ ID Nos: 3, 5, or 7.

12. The composition of any of claims 4-11, wherein the nucleic acid has at least 80%, at least 90%, at least 95%, or at least 99% sequence homology to SEQ ID Nos: 3 or 5.

13. The composition of any of claims 4-12, wherein the nucleic acid has at least 80%, at least 90%, at least 95%, or at least 99% sequence homology to SEQ ID No: 3.

14. The composition of any of claims 1-13, wherein the active agent comprises more than one antagomir, each comprising a nucleic acid that hybridizes to a different part of the miR17-92 cluster under moderate stringent conditions.

15. The composition of any of claims 1-14, wherein the one or more ionizable lipids, one or more cationic lipids, or a combination thereof are present in the lipid particle in an amount of from greater than 20 mol % to 75 mol %, based on the total components forming the lipid particle.

16. The composition of any of claims 1-15, wherein the one or more cationic lipids are present in the lipid particle in an amount of from greater than 0 mol % to 10 mol %, such as from 0.5 mol % to 5 mol % or from 4 mol % to 8 mol %, based on the total components forming the lipid particle.

17. The composition of any of claims 1-16, wherein the one or more cationic lipids comprise DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA (2,3-dioleyloxy-N-[2-(spermine carboxamido)ethyl]-N,N-dimethyl-1-propanaminium trifluoroacetate), DORIE (N-[1-(2,3-dioleyloxypropyl)]-N,N-dimethyl-N-hydroxyethylammonium bromide), DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimnethylammnonioacetyl)diethanolaninc chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammonium, oligofectamine, lipids described in U.S. Pat. No. 5,049,386, N-[1-(2,3-dioleyloxypropyl)]-N,N-dimethyl-N-hydroxyethylammonium bromide (DORIE), 2,3-dioleyloxy-N-[2-(spermine carboxamido)ethyl]-N,N-dimethyl-1-propanaminium trifluoroacetate (DOSPA), and the like as disclosed in International Publication Nos. WO91/16024 and WO97/019675; DLinDMA and the like as disclosed in International Publication No. WO2005/121348; and DLin-K-DMA and the like as disclosed in International Publication No. WO2009/086558; and (3R,4R)-3,4-bis((Z)-Hexadec-9-enyloxy)-1-methylpyrrolidine, and N-Methyl-N,N-bis(2-((Z)-octadec-6-enyloxy)ethyl)amine and the like as disclosed in International Publication No. WO2011/13636, or any combination thereof.

18. The composition of any of claims 1-17, wherein the one or more ionizable lipids are present in the lipid particle in an amount of from 20 mol % to 65 mol %, based on the total components forming the lipid particle.

19. The composition of any of claims 1-18, wherein the one or more ionizable lipids comprise N,N-dimethyl-2,3-dioleyloxypropylamine (DODMA), [(4-hydroxybutyl)azanediyl]di(hexane-6,1-diyl)bis(2-hexyldecanoate) (ALC-0315), 9-heptadecanyl 8-{(2-hydroxyethyl)[6-oxo-6-(undecyloxy)hexyl]amino}octanoate (SM-102), DLin-MC3-DMA, DLin-KC2-DMA, 1-(2,3-bis(((9Z,12Z)-octadeca-9,12-dien-1-yl)oxy)propyl)pyrrolidine (A066), or any combination thereof.

20. The composition of any of claims 1-19, wherein the one or more neutral lipids are present in the lipid particle in an amount of from 35 mol % to 80 mol %, based on the total components forming the lipid particle.

21. The composition of any of claims 1-20, wherein the one or more neutral lipids comprise dipalmitoylphosphatidylcholine (DPPC), dioleoylphosphatidylethanolamine (DOPE), palmitoyloleoylphosphatidylcholine (POPC), egg phosphatidylcholine (EPC), distearoylphosphatidylcholine (DSPC), cholesterol, or any combination thereof.

22. The composition of any of claims 1-21, wherein the one or more PEGylated lipids are present in the lipid particle in an amount of from greater than 0 mol % to 5 mol %, based on the total components forming the lipid particle.

23. The composition of any of claims 1-22, wherein the one or more PEGylated lipids comprise a PEG-ditetradecylacetamide, a PEG-myristoyl diglyceride, a PEG-diacylglycerol, a PEG dialkyloxypropyl, a PEG-phospholipid, a PEG-ceramide, PEG-DMG, PEG-DSPE, or any combinations thereof.

24. The composition of any of claims 1-23, wherein the one or more lipids comprising a microglial targeting agent are present in the lipid particle in an amount of from greater than 0 mol % to 10 mol %, such as from 0.5 mol % to 5 mol % or from 4 mol % to 8 mol %, based on the total components forming the lipid particle.

25. The composition of any of claims 1-24, wherein the microglial targeting agent comprises a carbohydrate.

26. The composition of any of claims 1-25, wherein the microglial targeting agent comprises mannose.

27. The composition of any of claims 1-26, wherein the one or more lipids comprising a microglial targeting agent comprise Man-PEG-DSPE, the structure of which is shown below 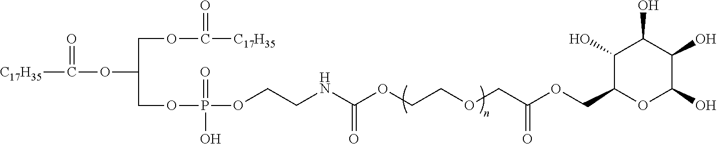
where n is an integer of from 1 to 1000.

28. The composition of any of claims 1-26, wherein the lipid particles comprise:
from greater than 0 mol % to 10 mol % of one or more lipids comprising a mannose moiety;
from greater than 20 mol % to 75 mol % of one or more ionizable lipids, one or more cationic lipids, or a combination thereof;
from 35 mol % to 80 mol % of one or more neutral lipids; and
from greater than 0 mol % to 5 mol % of one or more PEGylated lipids.

29. The composition of claim 28, wherein the one or more lipids comprising a mannose moiety comprise Man-PEG-DSPE.

30. The composition of any of claims 28-29, wherein the one or more ionizable lipids, one or more cationic lipids, or a combination thereof comprise DODMA, DLin-MC3-DMA, or a combination thereof.

31. The composition of any of claims 28-30, wherein the one or more neutral lipids comprise DSPC, DOPC, cholesterol, or a combination thereof.

32. The composition of any of claims 28-31, wherein the one or more PEGylated lipids comprise PEG-DMG, PEG-DSPE, or a combination thereof.

33. The composition of any of claims 1-32, wherein the lipid particles have an average diameter of less than 1 micron, such as from 50 nm to 750 nm, 50 nm to 250 nm, from 50 nm to 200 nm, from 50 nm to 150 nm, or from 50 nm to 100 nm.

34. The composition of any of claims 1-33, wherein the lipid particles have a polydispersity index (PDI) of less than 0.4.

35. A method of delivering an active agent to a microglia cell, the method comprising contacting the microglia cell with the composition of any of claims 1-34.

36. A method of delivering an active agent to a microglia cell in vivo, the method comprising administering to a subject the composition of any of claims 1-34.

37. A method of modulating autophagy activity in a subject, the method comprising administering to a subject the composition of any of claims 1-34.

38. A method of treating or preventing of a neurodegenerative disorder in a subject in need thereof, the method comprising administering to the subject a therapeutically effective amount of the composition of any of claims 1-34.

39. A method of reducing the expression of miR-17 in a subject having a neurodegenerative disorder, the method comprising administering to the subject a therapeutically effective amount of the composition of any of claims 1-34.

40. A method of reducing the expression of Amyloid beta (Aβ) in a subject having a neurodegenerative disorder, the method comprising administering to the subject a therapeutically effective amount of the composition of any of claims 1-34.

41. The method of any of claims 38-40, wherein the neurodegenerative disorder comprises Alzheimer's disease.
